(19) United States
(12) Patent Application Publication
(10) Pub. No.: US 2024/0099767 A1
(43) Pub. Date: Mar. 28, 2024

(54) MEDICAL DIAGNOSIS AND TREATMENT SYSTEM

(71) Applicant: Heartlander Surgical, Inc., Westwood, MA (US)

(72) Inventors: Dwight Meglan, Westwood, MA (US); Neil Pollack, Melbourn (GB); Matthew Hickey, Melbourn (GB); James Richardson, Melbourn (GB); Ryan Bardsley, Manchester, MA (US); Mark P. Ottensmeyer, Watertown, MA (US); R. Maxwell Flaherty, Topsfield, MA (US); J. Christopher Flaherty, Nottingham, NH (US)

(21) Appl. No.: 18/038,563

(22) PCT Filed: Dec. 3, 2021
(86) PCT No.: PCT/US2021/061733

§ 371 Date: May 24, 2023

Related U.S. Application Data
(60) Provisional application No. 63/120,813, filed on Dec. 3, 2020.

(51) Int. Cl. A61B 18/14

(52) U.S. Cl. CPC A61B 18/1492; A61B 2017/00199

(57) ABSTRACT

A system for treating and/or diagnosing tissue from a tissue surface comprises: a console; an interface assembly; and a mobile device. The mobile device is configured to navigate a tissue surface and can comprise: a first tissue attachment assembly; a second tissue attachment assembly; and at least one articulation assembly. The interface assembly operably connects the console to the mobile device. The console is configured to manipulate the at least one articulation assembly via the interface assembly. The at least one articulation assembly is configured to navigate from a first tissue location to a second tissue location. The mobile device is configured to diagnose and/or treat tissue at the second tissue location. Methods of treating and/or diagnosing tissue are also described.

### RELATED APPLICATIONS

This application claims priority to U.S. Provisional Patent Application Ser. No. 63/120,813 (Docket No. HLR-002-PR1), titled “Medical Diagnosis and Treatment System”, filed Dec. 3, 2020, the content of which is incorporated herein by reference in its entirety for all purposes.

This application is related to U.S. patent application Ser. No. 15/172,124 (Docket No. HLR-001-US), titled “Therapy Delivery System that Operates on the Surface of an Anatomical Entity”, filed Jun. 2, 2016, United States Publication Number US2016/0367312, published Dec. 22, 2016, the content of which is incorporated herein by reference in its entirety for all purposes.

### TECHNICAL FIELD

The embodiments disclosed herein relate generally to systems for performing a medical treatment on a patient, particularly systems that include a treatment component that can navigate along a tissue surface of the patient.

### BACKGROUND

An easier to use, less expensive, safer, more effective, and more widely applicable radiofrequency ablation (“RFA”) treatment for ventricular tachycardia (“VT”) is needed.

As reported in 2015, VT and related Ventricular Fibrillation (“VF”) are associated with increased morbidity and mortality and account for an estimated 63% of the 180 k-450 k annual cases of sudden cardiac death in the US. Antiarrhythmic drugs, the first line treatment, do not eliminate VT, can worsen symptoms, can be harmful to other organs, and are expensive for both patient and insurer. So, patients with life-threatening VT are frequently treated with an implantable cardioverter-defibrillator (“ICD”) that terminates but does not prevent VT. ICDs are expensive ($30,000 plus implantation cost), last at most 5-7 years, and provide limited overall mortality reduction of 1-3%. The possibly frequent shocks are painful, and are associated with anxiety and depression. Reducing ICD shock frequency is a major objective of ablation therapy.

RFA can eliminate the triggers and substrate for VT and reduce/prevent consequent ICD shocks in patients at high risk of sudden cardiac death. However, current RFA procedures are lengthy and require great technical skill, and so are only performed at select specialized centers. Precise ablation of the VT circuit typically requires activation, pace, or entrainment mapping that, in turn, typically requires prolonged maintenance of VT for the needed measurements. Since this is rarely possible in patients with hemodynamically untolerated VTs, precise RFA techniques are unsuitable for the 70-80% of VTs that are fast and/or hemodynamically unstable. Assist devices to enable these patients to receive RFA (e.g., Tandem Heart® or Impella®) are impractically expensive (about $20K) and may increase thrombus risk. The final alternative is for patients to undergo substrate mapping which typically requires a time consuming procedure done by highly specialized electrophysiologists (“EP”) needing to use rather large ablations to ensure success. Clearly, the majority of VT patients cannot benefit from the demonstrated quality of life improvements from RFA using current RFA techniques.

A typical RFA VT procedure costs around $20K in 2016; >55% of which is the cost of developing the electrophysiological map. Because high skill is required to manipulate an endocardial ablation catheter precisely, emboli and complication rates have been estimated to be 6-8%. More precise ablations may reduce the risk of subsequently developing new reentrant circuits via remodeling. Revision is required in 12-20% of cases within two years. So even in the hands of the most skilled, experienced EP, the current procedures’ success is less than it could be.

Thus, the combination of limitations in the currently addressable patient population and the difficulty in carrying out the procedure need to be transcended to bring RFA to the wider VT patient population.

### BRIEF SUMMARY

According to an aspect of the present inventive concepts, a system for treating and/or diagnosing tissue from a tissue surface, the system comprises: a console; an interface assembly; and a mobile device configured to navigate the tissue surface, the mobile device comprising: a first tissue attachment assembly; a second tissue attachment assembly; and at least one articulation assembly. The interface assembly operably connects the console to the mobile device. The console is configured to manipulate the at least one articulation assembly via the interface assembly. The at least one articulation assembly is configured to navigate from a first tissue location on the tissue surface to a second tissue location on the tissue surface by: at least one time performing a first movement by manipulating the position of the first tissue attachment assembly relative to the tissue surface while the second tissue attachment assembly is attached to the tissue; and at least one time performing a second movement by manipulating the position of the second tissue attachment assembly relative to the tissue surface while the first tissue attachment assembly is attached to tissue. The mobile device is configured to diagnose and/or treat tissue proximate the second tissue location.

In some embodiments, the at least one articulation assembly is configured to navigate from the first tissue location to the second tissue location by performing the first movement multiple times and by performing the second movement multiple times.

In some embodiments, the mobile device comprises at least one ultrasound transducer. The ultrasound transducer can be located within either the first or second tissue attachment assembly.

In some embodiments, the mobile device comprises a first chassis comprising a base chassis that includes the first tissue attachment assembly and a second chassis comprising a nose chassis that includes the second tissue attachment assembly. The at least one articulation assembly can be configured to translate the nose chassis relative to the base chassis. The nose chassis can be configured to pitch relative to the base chassis about an articulation point. The articulation point can comprise at least two articulation points.

In some embodiments, the first attachment assembly comprises a first housing attached to a first gripper and a second housing attached to a second gripper, and the first and second housings articulate relative to each other. The first and second housings can be biased to articulate toward the tissue surface.

In some embodiments, the mobile device further comprises a needle assembly comprising a needle. The needle assembly can further comprise a needle deployment mechanism configured to deploy the needle from the mobile device into tissue. The needle can be configured to deploy along an arched trajectory. The device can comprise a lead screw, and the needle deployment mechanism can comprise a nut configured to articulate along the lead screw. The mobile device can be configured to emit energy into tissue via the needle. The energy can comprise RF energy and/or energy configured to electroporate tissue. The system can further comprise an agent, and the needle can be configured to deliver the agent into tissue. The agent can comprise a regenerative substance.

In some embodiments, the first and/or the second tissue attachment assembly comprises a projection with a castellated profile.

In some embodiments, the first and/or the second tissue attachment assembly comprises a skirt configured to engage the tissue surface. The skirt can comprise a curled edge.

In some embodiments, the interface assembly comprises a single use component and a limited reuse component.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a system for treating and/or diagnosing tissue from a tissue surface, consistent with the present inventive concepts.

FIG. 2 illustrates a flowchart of a method of maneuvering a device to a desired tissue location, consistent with the present inventive concepts.

FIGS. 3A and 3B illustrate an exploded perspective view and a perspective view of a device, respectively, consistent with the present inventive concepts.

FIGS. 4A and 4B illustrate an exploded perspective view and a perspective view of a device, respectively, consistent with the present inventive concepts.

FIGS. 5A-C illustrate perspective views of a device in various deployment positions, consistent with the present inventive concepts.

FIG. 6 illustrates a schematic view of a system including a device for diagnosing and/or treating a patient, consistent with the present inventive concepts.

FIG. 7 illustrates a perspective view of a treatment device, consistent with the present inventive concepts.

FIGS. 8A-C illustrate three articulated orientations of a device, consistent with the present inventive concepts.

FIGS. 9A and 9B illustrate a bottom and a side view of a portion of an attachment assembly, respectively, consistent with the present inventive concepts.

FIGS. 10A-C illustrate three articulated orientations of a device, consistent with the present inventive concepts.

FIGS. 11A-C illustrate three configurations of a device illustrating forward locomotion of the device, consistent with the present inventive concepts.

FIGS. 12A-C and 13A-C illustrate three side and three top views, respectively, of a needle deployment assembly, consistent with the present inventive concepts.

FIG. 14 illustrates a perspective view of the underside of the nose chassis of a treatment device, consistent with the present inventive concepts.

FIG. 15 illustrates a top view of a treatment device, consistent with the present inventive concepts.

FIGS. 16A-C illustrate a back-perspective view, a top view, and a front-perspective view, respectively, of a treatment device, consistent with the present inventive concepts.

FIGS. 17A-C illustrate a side-perspective view, a bottom-perspective view, and a front-perspective view, respectively, of another embodiment of a treatment device, consistent with the present inventive concepts.

FIG. 18 illustrates a schematic view of a treatment system illustrating portions of a treatment device, interface assembly, and console, consistent with the present inventive concepts.

FIG. 19 illustrates a flowchart of a method of maneuvering a device to a desired tissue location, consistent with the present inventive concepts.

FIGS. 20A-0 illustrate various views of various embodiments of a treatment device, consistent with the present inventive concepts.

FIGS. 21A and 21B illustrate perspective sectional views of various embodiments of a gripper for securing a treatment device to tissue, consistent with the present inventive concepts.

FIG. 22 illustrates a sectional view of a tissue attachment assembly, consistent with the present inventive concepts.

FIG. 23 illustrates a sectional view of a tissue attachment assembly, consistent with the present inventive concepts.

FIGS. 24A and 24B illustrate sectional views of a tissue attachment assembly, consistent with the present inventive concepts.

FIGS. 25A and 25B illustrate sectional views of a tissue attachment assembly, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

It will be understood that the words “comprising” (and any form of comprising, such as “comprise” and “comprises”), “having” (and any form of having, such as “have” and “has”), “including” (and any form of including, such as “includes” and “include”) and/or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of two or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, “prevention” and the like, where used herein, shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. “Positive pressure” includes pressure above room pressure or simply a pressure that is greater than another pressure, such as a positive differential pressure across a fluid pathway component such as a valve. “Negative pressure” includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “transducer” is to be taken to include any component or combination of components that receives energy or any input and produces an output. For example, a transducer can include an electrode that receives electrical energy and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: heat energy to tissue; cryogenic energy to tissue; electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of two or more of these. Alternatively or additionally, a transducer can comprise a mechanism, such as: a valve; a grasping element; an anchoring mechanism; an electrically-activated mechanism; a mechanically-activated mechanism; and/or a thermally activated mechanism.

As used herein, the term “functional element” is to be taken to include one or more elements constructed and arranged to perform a function. A functional element can comprise one or more sensors and/or one or more transducers. In some embodiments, a functional element is configured to deliver energy and/or otherwise treat tissue (e.g. a functional element configured as a treatment element). Alternatively or additionally, a functional element (e.g. comprising one or more sensors) can be configured to record one or more parameters, such as a patient physiologic parameter; a patient anatomical parameter (e.g. a tissue parameter); a patient environment parameter; and/or a system parameter (e.g. temperature and/or pressure within the system). In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a patient anatomical parameter; and combinations of two or more of these. A “functional assembly” can comprise an assembly constructed and arranged to perform a function, such as is described hereabove. In some embodiments, a functional assembly is configured to deliver energy and/or otherwise treat tissue (e.g. a functional assembly configured as a treatment assembly). Alternatively or additionally, a functional assembly can be configured to record one or more parameters, such as a patient physiologic parameter; a patient anatomical parameter; a patient environment parameter; and/or a system parameter. A functional assembly can comprise an expandable assembly and/or an otherwise deployable assembly. A functional assembly can comprise one or more functional elements.

As used herein, the term “agent” shall include but not be limited to one or more agents selected from the group consisting of: an agent configured to improve and/or maintain the health of a patient; a drug (e.g. a pharmaceutical drug); a hormone; a protein; a protein derivative; a small molecule; an antibody; an antibody derivative; an excipient; a reagent; a buffer; a vitamin; a nutraceutical; and combinations of these.

As used herein, the term “conduit” or “conduits” can refer to an elongate component that can include one or more flexible and/or non-flexible filaments selected from the group consisting of: one, two or more wires or other electrical conductors (e.g. including an outer insulator); one, two or more wave guides; one, two, or more hollow tubes, such as hydraulic, pneumatic, and/or other fluid delivery tubes; one or more optical fibers; one two or more control cables, torque cables, and/or other mechanical linkages; one, two or more flex circuits; and combinations of these. A conduit can include a tube including multiple conduits positioned within the tube. A conduit can be configured to electrically, fluidically, sonically, optically, mechanically, and/or otherwise operably connect one component to another component.

Provided herein are systems for performing a treating and/or diagnosing tissue of a patient. The systems can include a mobile device configured to navigate a tissue surface (e.g. an endocardial and/or epicardial surface of the heart). The mobile devices can include various assemblies for navigating from location to location on the tissue surface, such as to perform a medical procedure at one or more of these tissue surface locations. A console can be included that can manipulate one or more portions of the navigation assemblies.

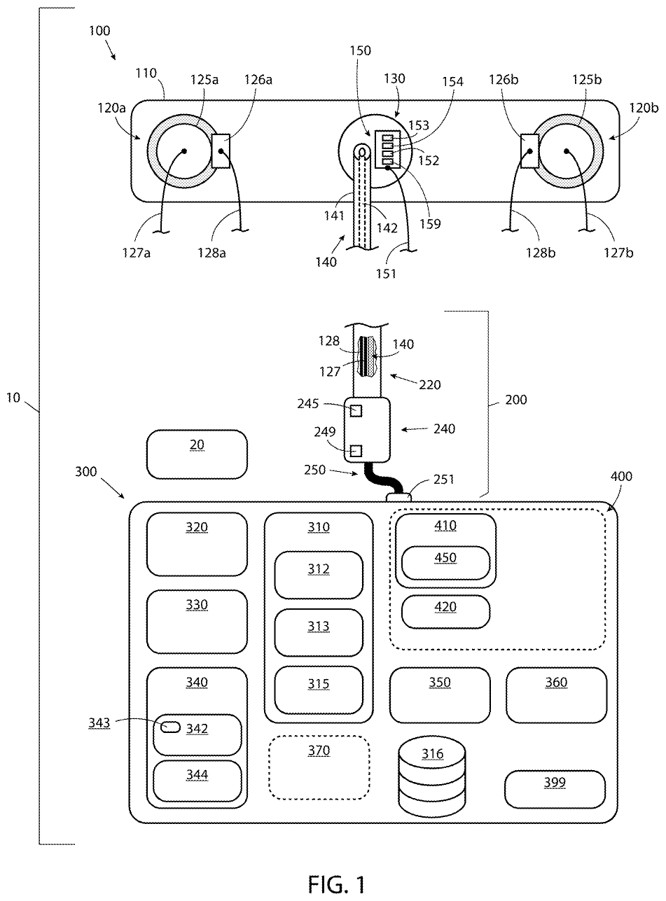

Referring now to FIG. 1, a system for treating and/or diagnosing (“treating” herein) tissue from a tissue surface is illustrated, consistent with the present inventive concepts. System 10 includes device 100 that operably attaches to console 300 (e.g. electrically, mechanically, optically, fluidically, pneumatically, hydraulically, sonically, and/or otherwise operatively attaches to console 300). In some embodiments, device 100 attaches to console 300 via interface assembly 200. System 10 and device 100 can be used by an operator (e.g. one or more clinicians) to perform a clinical procedure (e.g. a therapeutic procedure and/or a diagnostic procedure) on a patient. Device 100 comprises a mobile device that can be configured as a medical treatment and/or diagnosis device (“treatment device” herein). Device 100 can be constructed and arranged to maneuver along a tissue surface (e.g. a surface of a body organ), for example, an epicardial and/or endocardial surface of the heart. Device 100 includes hub assembly 130 comprising one or more therapeutic and/or diagnostic elements (“treatment elements” herein). Device 100 can be maneuvered along the tissue surface (e.g. maneuvered in response to one or more commands received from console 300), such as to position hub assembly 130 proximate a tissue location (“treatment location” herein) at which a “treatment” is performed (e.g. one or more diagnostic treatments and/or therapeutic treatments are performed). For example, device 100 can be maneuvered to position hub assembly 130 on or otherwise proximate the treatment location, such that one or more of the treatment elements of hub assembly 130 described herein are aligned with the treatment location.

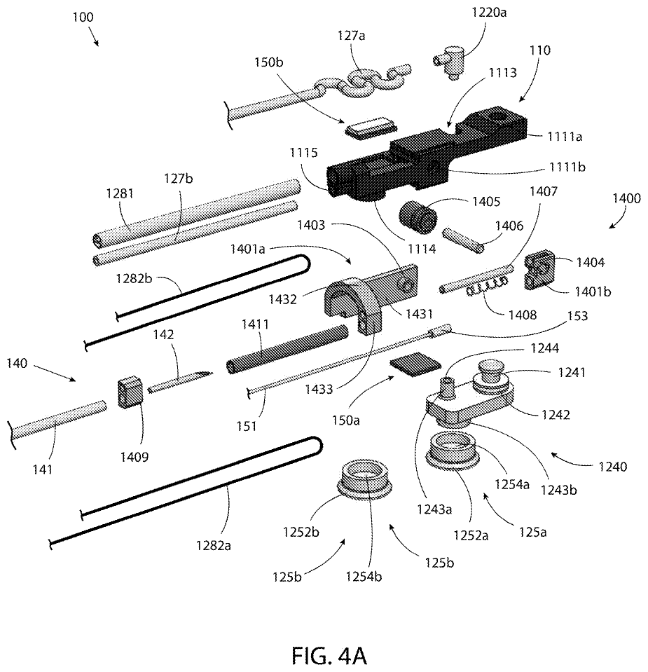

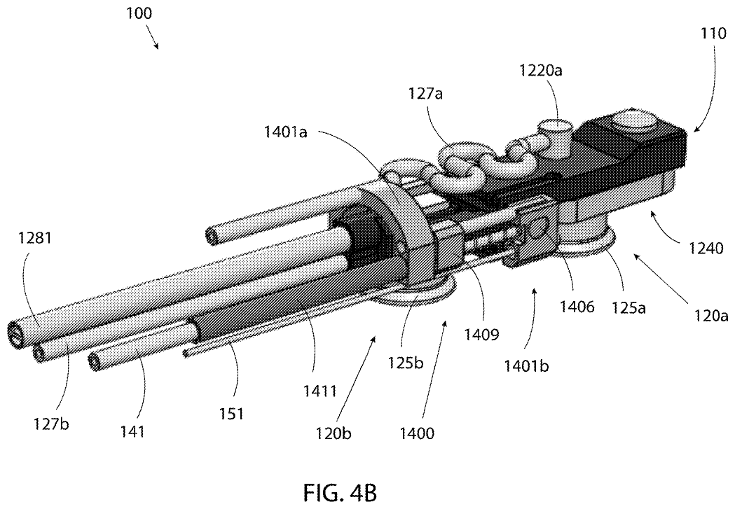

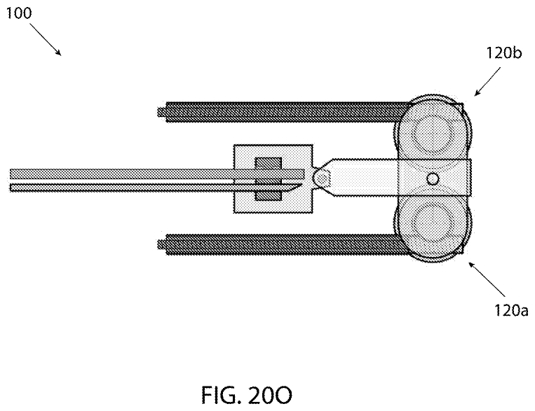

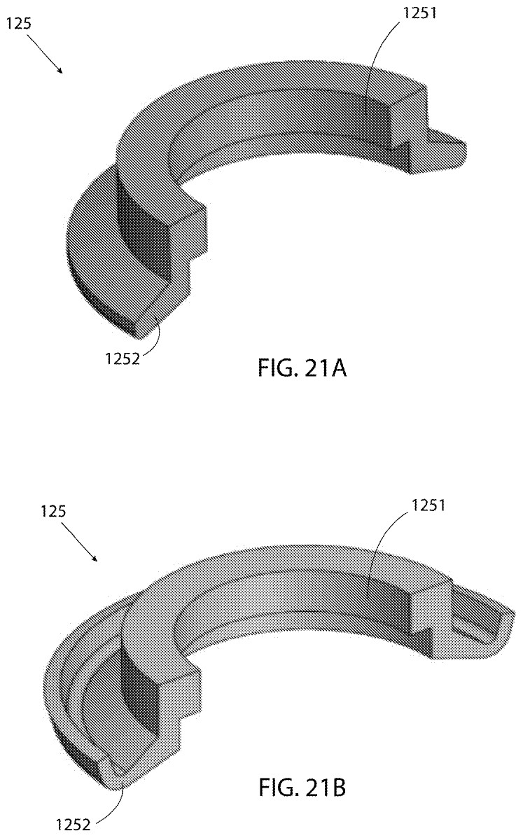

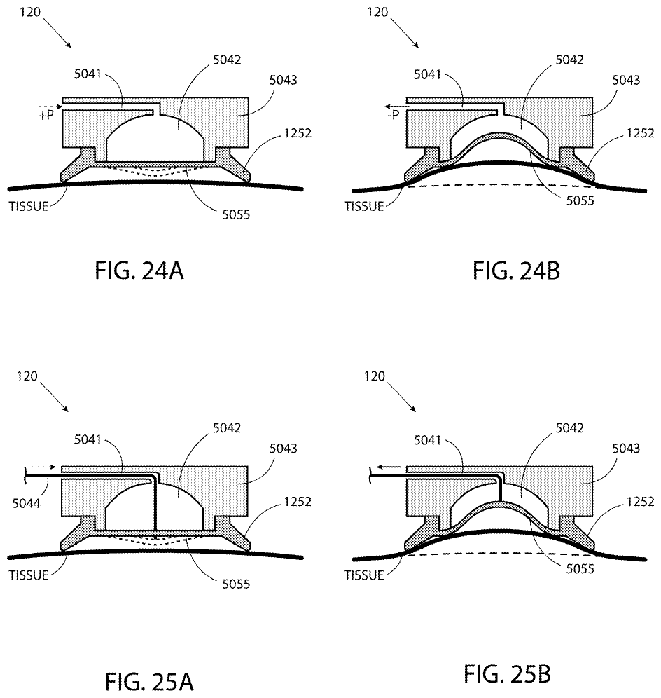

Device 100 comprises one or more chassis, chassis 110 shown. Device 100 can include two or more mechanisms for securely attaching to (e.g. engaging with) and detaching from (e.g. disengaging with) a tissue surface, tissue attachment assembly 120 (e.g. assemblies 120a and 120b shown). Each tissue attachment assembly 120 includes a mechanism for the tissue attachment and detachment, gripper 125 (e.g. grippers 125a and 125b shown). In some embodiments, gripper 125 comprises a suction-based mechanism (e.g. a suction cup-like mechanism) that attaches to the tissue surface using its applied suction. In some embodiments, tissue attachment assembly 120 further comprises a conduit, attachment conduit 127 (e.g. conduits 127a and 127b shown). Attachment conduit 127 can be configured to operably connect gripper 125 to console 300 (e.g. via interface assembly 200), such as to allow console 300 to attach and/or detach gripper 125 to and/or from the tissue surface. For example, conduit 127 can operably attach attachment assembly 120 to a source of vacuum to provide a suction force to gripper 125. Alternatively or additionally, conduit 127 can comprise a control wire (not shown) configured to actuate an electronic portion of gripper 125, such as to electrically manipulate a suction chamber within gripper 125. In some embodiments, gripper 125 can comprise a membrane (e.g. membrane 1254 of FIG. 4A and/or membrane 5055 of FIGS. 24A-25B) configured to be moved (e.g. pulled) away from tissue to create a suction force between gripper 125 and tissue (e.g. similar to a suction cup). In some embodiments, the membrane is moved via a physical manipulation and/or via the application of a vacuum. Various implementations of gripper 125 are described herein in reference to FIGS. 21A-25B.

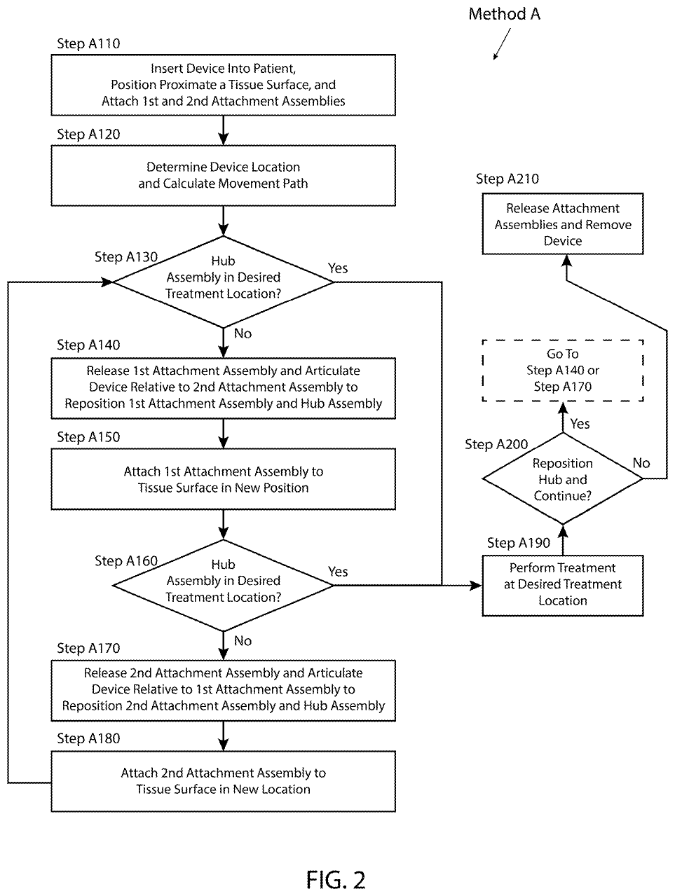

In some embodiments, each tissue attachment assembly 120 is rotatably attached to chassis 110 (e.g. such that chassis 110 can rotate about an assembly 120 when that assembly 120 is attached to tissue). Each tissue attachment assembly 120 can comprise a rotational control mechanism, rotation control assembly 126 (e.g. assemblies 126a and 126b shown). Rotation control assembly 126 can manipulate the relative angular orientation between tissue attachment assembly 120 and chassis 110. For example, if first tissue attachment assembly 120a is attached to the tissue surface and second tissue attachment assembly 120b is detached from the tissue surface, rotation control assembly 126a can be actuated to rotate chassis 110 relative to the tissue surface about a central axis of tissue attachment assembly 120a. Device 100 can be configured to maneuver along a tissue surface by rotating, in an alternating fashion, about first tissue attachment assembly 120a and then second tissue attachment assembly 120b, such as is described in reference to FIG. 2 herein. Rotation control assembly 126 can include a conduit, control conduit 128 (e.g. conduits 128a and 128b shown), that operably connects rotation control assembly 126 to console 300 (e.g. via interface assembly 200). In some embodiments, control conduit 128 comprises a wire (e.g. a torque wire) which can be configured to transfer torsional force (i.e. to transfer torque) from console 300 to rotation control assembly 126. Additionally or alternatively, control conduit 128 can be configured to transfer a pushing and/or a pulling force to control assembly 126.

Device 100 can comprise one or more assemblies for advancing a needle into tissue proximate hub assembly 130, needle assembly 140 shown. Needle assembly 140 can comprise a tube with one or more lumens, and/or other components including a passageway, conduit 141. A needle or a tube with a needle on its distal end, needle 142 shown, can be configured to translate within conduit 141. Alternatively, conduit 141 can comprise needle 142 on its distal end, and conduit 141 can be configured to translate (e.g. advance and/or retract relative to hub assembly 130). Needle 142 can comprise an elongate, hollow structure, with a sharpened distal end. System 10, via needle 142, can be configured to deliver a substance (e.g. a drug or other agent, agent 343 shown) to tissue, such as to perform a therapeutic and/or diagnostic procedure. Alternatively or additionally, system 10 can be configured to perform a biopsy (e.g. using needle 142 to collect a sample of tissue).

Device 100 can comprise one or more electronic assemblies, electronics assembly 150. In some embodiments, at least a portion of electronics assembly 150 is positioned within hub assembly 130, as shown. Electronics assembly 150 can comprise one or more sensors, transducers, functional elements, circuit boards, power supplies, and/or other components configured to enable and/or perform one or more functions of device 100. For example, electronics assembly 150 can include one or more ultrasound transducers, ultrasound transducer 153. Ultrasound transducer 153 can be configured to emit and/or receive ultrasonic pulses into and/or from tissue (e.g. target tissue), such as to image a portion of the tissue and/or deliver ultrasonic energy into tissue. In some embodiments, system 10 is configured to employ Doppler ultrasound via ultrasound transducer 153, for example, to detect the presence of a blood vessel within the target tissue (e.g. by detecting flow of blood proximate hub assembly 130 and/or ultrasound transducer 153). In some embodiments, an algorithm of system 10 (e.g. algorithm 315 described herein) is configured to analyze Doppler ultrasound images to determine the presence of a blood vessel. In some embodiments, electronics assembly 150 comprises one or more sensors, sensor 152 shown. Sensor 152 can comprise a sensor selected from the group consisting of: temperature sensor; pressure sensor; strain gauge; accelerometer; pH sensor; acoustic sensor; chemical sensor; electrode; electromagnetic sensor; flow sensor; blood pressure sensor; blood gas sensor; blood glucose sensor; heart rate sensor; respiration sensor; optical spectroscopy sensor; electrical impedance spectroscopy sensor; and combinations of these. Electronics assembly 150 can include one or more functional elements, functional element 159. Functional element 159 can comprise a functional element selected from the group consisting of: sensor; transducer; vibrational element; heating element; cooling element; light emitting element; sound emitting element; agent delivery element; needle; an electrode, such as an electrode configured to emit an electrical current; and combinations of these. In some embodiments, electronics assembly 150 includes one or more conduits, communication conduit 151, for transmitting power and/or data to and/or from electronics assembly 150. For example, communication conduit 151 can operably attach to console 300 (e.g. via interface assembly 200) to transmit information between console 300 and electronics assembly 150. Electronics assembly 150 can include one or more electrodes, electrode 154. Electrode 154 can be configured to emit electrical energy (e.g. RF energy) and/or to record electrical signals (e.g. cardiac signals), as described herein.

In some embodiments, interface assembly 200 operably attaches one or more components of device 100 to console 300. Alternatively or additionally, one or more components of device 100 can attach directly to console 300, such as via a conduit 127 described herein. Interface assembly 200 can comprise one or more shafts, shaft assembly 220. Shaft assembly 220 can comprise a hollow shaft that surrounds one or more conduits extending from device 100 to console 300. For example, shaft assembly 220 can surround at least a portion of one or more of: attachment conduits 127; control conduits 128; needle assembly 140; communication conduit 151; other conduits or elongate assemblies extending from device 100; and combinations of these. Alternatively or additionally, shaft assembly 220 can comprise one or more aligning elements configured to attach to one or more locations along the lengths of two or more of the conduits that extend from device 100 to console 300. These aligning elements can be included to maintain the arrangement (e.g. circumferential arrangement) and/or alignment of the conduits along their length (e.g. without encasing the conduits with an outer shaft). In some embodiments, the aligning elements are positioned along the conduits such that the bending stiffness of shaft assembly 220 is minimally affected (e.g. minimally increased) by the connectors. In some embodiments, interface assembly 200 comprises a connector assembly 250. Connector assembly 250 can comprise one or more connectors, connector 251 shown, that operably attaches one or more conduits and/or other assemblies that extend through shaft assembly 220 to console 300. In some embodiments, connector 251 comprises a connector configured to operably attach two or more conduits with a single connection, such as to limit the number of connections that are required (e.g. connections made by an operator of system 10 during and/or before a clinical procedure) to properly attach device 100 to console 300.

In some embodiments, interface assembly 200 comprises a handheld control assembly, handle assembly 240. Handle assembly 240 can be positioned along or proximate shaft assembly 220, for example, at the proximal end of shaft assembly 220, with connector assembly 250 extending from handle assembly 240 (as shown). Handle assembly 240 can comprise one or more controls, control 245 shown. Control 245 can be configured to allow an operator to manipulate device 100. For example, control 245 can provide the operator with an interface to initiate and/or otherwise control an action to be performed by device 100. For example, a series of actions can be enabled by console 300 and performed using device 100, where one or more of these actions are initiated based on operator input via control 245 (e.g. operator activation of a “go” button portion of control 245). Interface assembly 200 can comprise one or more functional elements (e.g. as described herein), such as functional element 249 shown. Alternatively, in some embodiments, interface assembly 200 does not include a handle assembly 240, for example when shaft assembly 220 extends directly from device 100 to console 300. In these embodiments, system 10 can comprise a user input for controlling device 100, for example user input 420 described herebelow.

Console 300 can comprise processing unit 310 shown. Processing unit 310 can comprise one or more electronic elements, electronic assemblies, and/or other electronic components, such as components selected from the group consisting of: memory storage components; analog-to-digital converters; rectification circuitry; state machines; microprocessors; microcontrollers; filters and other signal conditioners; sensor interface circuitry; transducer interface circuitry; and combinations thereof. Processing unit 310 can comprise at least one microprocessor, computer, and/or other electronic controller, processor 312 shown. Processing unit 310 can also include memory 313. Memory 313 can include high-speed random-access memory and/or non-volatile memory, such as one or more magnetic disk, optical, and/or flash memory storage devices. In some embodiments, memory 313 includes instructions, such as instructions used by processing unit 310 to perform one or more algorithms, such as algorithm 315 shown. Algorithm 315 can comprise one or more algorithms that are configured to analyze data (e.g. data produced by a sensor-based functional element of system 10) and produce a result. System 10 can include an interface, user interface 400, for providing and/or receiving information to and/or from an operator of system 10. User interface 400 can be integrated into console 300 as shown. In some embodiments some or all of user interface 400 can comprise a component separate from console 300, such as a monitor separate from, but operably attached to, console 300. User interface 400 can include one, two, or more user input and/or user output components. For example, user interface 400 can comprise a joystick, keyboard, mouse, touchscreen, and/or other human interface device, user input device 420 shown. In some embodiments, user interface 400 comprises a display (e.g. a touchscreen display), such as display 410, also shown. In some embodiments, processor 312 can provide a graphical user interface, GUI 450, to be presented on display 410. In some embodiments, user interface 400 comprises a device of similar construction and arrangement to a video game controller, such as a device providing one and/or two-handed control of device 100, and including multiple control inputs including buttons, triggers, and/or joysticks.

In some embodiments, system 10 comprises additional volatile and/or non-volatile memory, data storage 316. Data storage 316 can be configured to store data collected by system 10 during one or more clinical procedures, such as image data described herein. In some embodiments, data storage 316 can comprise cloud-based storage, for example, when console 300 is connected to a local and/or a wide area network, such as the Internet. In some embodiments, data storage 316 includes data collected from multiple patients over multiple procedures (e.g. multiple procedures each using system 10). In some embodiments, algorithm 315 comprises a machine learning or other artificial intelligence algorithm, such as an algorithm configured to analyze the data collected from system 10 (e.g. data collected from multiple patients and/or simulated data generated in testing, manufacturing, and/or the development of system 10), and to produce additional data from that analysis. For example, algorithm 315 can comprise an artificial intelligence algorithm that is configured to produce data relating to the health of a patient, relating to a diagnostic and/or a prognostic decision, relating to suggested procedural steps (e.g. a suggested movement path of device 100 along the tissue surface), or other data determined by identifying patterns in previously and/or currently collected data.

Algorithm 315 can be configured to determine a movement path along a tissue surface to advance device 100 from a current location to a desired treatment location. The movement path can comprise multiple “footsteps”, or sequential target locations to place the first and second attachment assemblies 120a,b in an alternating fashion to move toward the desired treatment location. Algorithm 315 can be configured to determine the location of the path footsteps based on a set of movement rules, such as the distance between each footstep, the maximum angle of rotation about each attachment assembly 120, the shortest path comprising the least number of footsteps, and/or other rules based on: the parameters of device 100 and/or other components of system 10; operator input and/or other operator preferences; and/or patient anatomy and/or other patient parameters. In some embodiments, patient parameters include the location of the origination and/or propagation site of an arrhythmia. In some embodiments, algorithm 315 applies a “safety margin” to a calculation to be performed. For example, algorithm 315 can be configured to avoid (e.g. for some or all of a set of path footsteps, and/or for an eventual target location) certain anatomical locations, “avoidance locations” herein, and algorithm 315 can avoid these avoidance locations by a particular safety margin distance, such as a safety margin distance of at least 1 mm, 2 mm, 3 mm, and/or 5 mm. In some embodiments, algorithm 315 is configured to create a set of path footsteps and/or an eventual target location that are positioned relatively equidistantly between two or more avoidance locations. In some embodiments, algorithm 315 includes a bias, such as a bias towards certain target tissue and/or away from certain avoidance locations. In some embodiments, algorithm 315 comprises a bias configured to tend to perform one or more of a set of motions, and to tend to avoid one or more other sets of motions. For example, to tend to perform a motion including a rotation about each attachment assembly 130 under a threshold angle of rotation, while avoiding similar rotations above that threshold.

System 10 can include one or more imaging devices, imaging device 370 shown. Imaging device 370 can be integral to and/or separate from and configured to interface with console 300. In some embodiments, imaging device 370 comprises two or more imaging devices, for example, a fluoroscopic imaging device and an Mill device. In these embodiments, console 300 can be configured to receive image data from imaging devices 370, such as to display the image data on display 410. Imaging device 370 can comprise one or more imaging devices selected from the group consisting of: a fluoroscope or other X-ray imaging device; a CT Scanner; an Mill device; an ultrasonic imaging device; a visual imaging device, such as a visible light camera; an infrared imaging device, such as an infrared camera; and combinations of these. In some embodiments, functional element 159 of electronics assembly 150 comprises an imaging device, such as a camera, and imaging device 370 is configured to operably attach to functional element 159 (e.g. via communication conduit 151) to receive and/or process image data captured by functional element 159. In some embodiments, functional element 159 comprises a lens assembly, and communication conduit 151 comprises a fiber optic cable configured to transmit light captured by functional element 159 to a camera component or imaging device 370, such as to produce image data from the received light. Alternatively or additionally, conduit 151 can transmit light to functional element 159 such that light is emitted from functional element 159 comprising a lens assembly. In some embodiments, imaging device 370 is configured to provide a source of light to be transmitted to functional element 159. In some embodiments, the light source is configured to provide light at one or more specific wavelengths (e.g. one or more infrared and/or visible wavelengths). In some embodiments, functional element 159 further comprises a light emitting element, such as an LED, which can be configured to illuminate tissue to increase the amount of light that can be captured by a functional element 159 comprising a lens assembly. In some embodiments, functional element 159 comprises an image sensor, such as a CCD image sensor. In some embodiments, functional element 159 comprises a camera assembly, such as an assembly including a lens and an image sensor. In some embodiments, functional element 159 comprises an actuator configured to manipulate the camera assembly, such as to optically zoom and/or to change the orientation of the camera assembly relative to device 100 to change the field of view of the camera relative to device 100.

Console 300 can include a set of one or more components, attachment control module 320, for interfacing with one or more tissue attachment assemblies 120 of device 100. Attachment control module 320 can comprise one or more motors, pneumatic controllers, fluidic controllers, actuators, or other components configured to control one or more functions of a tissue attachment assembly 120 of device 100. In some embodiments, attachment control module 320 is operably connected to a tissue attachment assembly 120 via one more attachment conduits 127 (e.g. via interface assembly 200). Attachment control module 320 can be configured to independently manipulate two or more tissue attachment assemblies 120. For example, attachment control module 320 can be configured to cause tissue attachment assembly 120a to attach to tissue, and simultaneously cause tissue attachment assembly 120b to detach from tissue. In some embodiments, attachment control module 320 provides a source of vacuum to a tissue attachment assembly 120, such as to provide a suction force to gripper 125 of attachment assembly 120. In other embodiments, attachment control module 320 is configured to manipulate a linkage (e.g. when conduit 127 comprises a linkage), such as to provide a relative translational force within tissue attachment assembly 120 to actuate a portion of gripper 125, for example, when gripper 125 comprises a suction cup type construction which requires a force to be applied to form a suction based attachment.

Console 300 can include a set of one or more components, articulation control module 330, for interfacing with one or more rotation control assemblies 126 of device 100. Articulation control module 330 can comprise one or more motors, pneumatic controllers, fluidic controllers, actuators, and/or other components configured to interface with a rotation control assembly 126 of device 100. In some embodiments, articulation control module 330 is operably connected to a rotation control assembly 126 via one or more control conduits 128 (e.g. via interface assembly 200). Articulation control module 330 can be configured to independently manipulate two or more rotation control assemblies 126. For example, articulation control module 330 can be configured to cause rotation of a portion of tissue attachment assembly 120a in a first direction (e.g. a clockwise direction) relative to chassis 110, while simultaneously preventing rotation (e.g. preventing any rotation) of a component of tissue attachment assembly 120b with respect to chassis 110.

Console 300 can include a set of one or more components, needle control module 340, for interfacing with needle assembly 140. Needle control module 340 can include an injection module 342 and/or a biopsy module 344. Injection module 342 can comprise a module that is configured to inject one or more substances, agent 343, into tissue via needle 142. Injection module 342 can comprise a user controllable injection device, such as a syringe, or a motorized injection device, such as a syringe pump. In some embodiments, agent 343 comprises two or more agents (e.g. two or more pharmaceutical agents). Injection module 342 can be configured to selectively inject a single agent from a group of two or more agents comprising agent 343. Biopsy module 344 can comprise a module that is configured to actuate or otherwise cause needle 142 to engage tissue to perform a biopsy. In some embodiments, biopsy module 344 comprises a vacuum or other mechanism for drawing a tissue sample through needle 142 into console 300. In some embodiments, needle 142 can be used to remove diseased or otherwise undesirable tissue from a patient via biopsy module 344. In some embodiments, needle control module 340 is configured to coordinate (e.g. synchronize) the needle insertion, needle retraction, injection of one or more substances (e.g. via one or more syringe pumps, such as syringe pump 321 not shown but described herein), biopsy suction, and/or other mechanism activations and/or functions of device 100 with the cardiac cycle. In some embodiments the cardiac cycle is assessed (e.g. determined) from signals provided by one or more sensors of device 100 (e.g. sensors 152, 153, 154, 170, and/or 171) and/or signals provided by one or more external sensors (e.g. one or more functional elements 399 configured as an external sensor).

Console 300 can include an interface, sensor interface 350, for communicating with one or more sensors of device 100 and/or for communicating with one or more sensors external device 100. Sensor interface 350 can be operably connected (e.g. at least electrically connected) to one or more sensors via communication conduit 151 (e.g. via interface assembly 200). In some embodiments, sensor interface 350 comprises hardware configured to enable ultrasound transducer 153 to perform one or more ultrasonic functions, such as those described herein (e.g. Doppler ultrasound imaging). Sensor interface 350 can be configured to interface with one or more sensors of system 10, such as when sensor 152 or another sensor of system 10 comprises one, two or more sensors selected from the group consisting of: electrode; ultrasonic transducer; blood sensor; blood glucose sensor; pH sensor; pressure sensor; blood pressure sensor; respiration sensor; motion sensor; strain gauge; optical sensor; magnetic sensor; and combinations of these.

Console 300 can include one or more components, energy delivery module 360, for delivering energy to tissue (e.g. via electrode 154). In some embodiments, energy delivery module 360 is configured to deliver ablative energy to tissue, such as RF energy sufficient to ablate tissue, via electrode 154 of electronics assembly 150. Additionally or alternatively, energy delivery module 360 can be configured to deliver an energy pulse configured to reversibly and/or irreversibly electroporate tissue. Electrode 154 can comprise one or more electrodes configured to emit the energy into the tissue. In some embodiments, electrode 154 comprises two or more electrodes, such that bipolar RF energy can be delivered. Alternatively or additionally, a functional element of console 300 (e.g. functional element 399 described herein) can comprise an electrode patch, which can be configured to attach to the skin of the patient and provide an energy return path (e.g. for monopolar delivery of RF energy by device 100). In some embodiments, energy delivery module 360 is configured to emit the energy into the tissue via needle 142 (e.g. when electrode 154 is positioned on a portion of needle 142, and/or when needle 142 is electrically connected to energy delivery module 360).

In some embodiments, system 10 is configured to allow an operator to identify and/or treat (e.g. ablate) diseased tissue in which arrhythmogenic and/or other aberrant cardiac conduction pathways and/or dysfunctional myocardial contraction exist. Device 100 can be configured to maneuver along the epicardial surface of the heart and record biopotential signals correlating to the electrical activity of the heart, such as via electrode 154. In some embodiments, console 300 is configured to generate one or more maps of the cardiac electrical activity, based at least in part on these recorded biopotential signals. Additionally or alternatively, device 100 can be configured to maneuver along the epicardial surface and record signals indicating the presence of coronary vessels, pericardial adhesions, and/or myocardial scar tissue. In some embodiments, console 300 is configured to generate one or more maps (e.g. anatomical maps) of these and other heart features. In some embodiments, console 300 is configured to record image data from imaging device 370, such as when imaging device 370 produces magnetic resonance (MR) image data or a computerized tomography (CT) scan data. Console 300 can be further configured to co-register this image data with one or more maps (e.g. biopotential and/or anatomical maps) generated from the signals collected by device 100, such as to generate a new map. In some embodiments, console 300 generates an integrated map that contains information from one or more of the maps described above. Based on these one or more maps, diseased tissue can be identified, either by an operator of system 10, and/or automatically or semi-automatically by system 10, such as via an analysis performed by algorithm 315 of console 300. In some embodiments, algorithm 315 is configured to identify diseased tissue, and the information is displayed to the operator on display 410 via GUI 450. For example, GUI 450 can display an anatomic model of the heart, onto which one or more maps of cardiac electrical activity are displayed, and with which diseased tissue can be identified (e.g. by system 10 and/or by the operator, such as for subsequent treatment). In some embodiments, device 100 is configured to maneuver such that electronics assembly 150 (including electrode 154) is positioned in such a way that electrode 154 can be used to ablate (e.g. cause necrosis of) the diseased tissue via the application of electric energy.

In some embodiments, system 10 is configured to deliver one or more biological materials into tissue, such as when agent 343 comprises one or more biological substances. For example, agent 343 can comprise stem cells to be implanted into the patient. In some embodiments, system 10 can be configured to inject or otherwise implant stem cells proximate the site of an infarct of heart tissue. Device 100 can be maneuvered to tissue on the periphery of an infarct, and inject an agent 343 comprising stem cells into the tissue of the heart proximate the periphery (e.g. into the infracted tissue and/or into healthy tissue proximate the infarct). Alternatively or additionally, device 100 can be configured to harvest healthy tissue (e.g. using biopsy module 344) and implant the harvested tissue proximate the periphery of an infarct (e.g. into the infarcted tissue and/or into healthy tissue proximate the infract).

In some embodiments, agent 343 comprises one or more exosomes configured to provide a therapeutic benefit.

In some embodiments, agent 343 comprises one or more gene therapy agents.

In some embodiments, agent 343 comprises one or more regenerative substances, such as an angiogenic factor.

In some embodiments, agent 343 comprises optogenetic sensitive cells. Device 100 can be configured to implant these cells into heart tissue, for example, to allow biologically-based cardiac pacing and/or defibrillation via these implanted cells.

In some embodiments, agent 343 comprises immune cells (e.g. immune cells harvested from the patient and/or cells from a donor). Device 100 can be configured to implant these cells into heart tissue, for example to stabilize the cardiac rhythm of the patient.

In some embodiments, agent 343 comprises one or more drugs. For example, agent 343 can comprise one or more drugs configured to be injected into the heart tissue to minimize myocardial reperfusion injury. Device 100 can be configured to inject these drugs directly into the heart tissue (e.g. via needle assembly 140).

In some embodiments, system 10 includes a device configured to be chronically and/or temporarily implanted into the patient, implant 20. Device 100 can be configured to precisely position at least a portion of implant 20 for implantation, such as to position implant 20 within 5 mm, 3 mm, 2 mm, and/or 1 mm from a desired implant location. For example, device 100 can operably attach to a portion of implant 20, maneuver along a tissue surface to an implant location (e.g. as described herein), and release implant 20 at the implant location. In some embodiments, device 100 is configured to secure at least a portion of implant 20 to the tissue, for example, by embedding a portion of implant 20 within the tissue (e.g. using needle assembly 140). In some embodiments, implant 20 comprises one or more epicardial leads (e.g. pacing leads), and device 100 is configured to implant the one or more leads into the tissue of the heart in an arrangement configured for delivering biventricular pacing. Alternatively or additionally, device 100 can be configured to implant the implant 20 comprising one or more leads into the tissue of the heart proximate the HIS bundle. In some embodiments, implant 20 comprises a light emitting element (e.g. and LED), such as a light emitting element configured to trigger optogenetic sensitive cells, for example when agent 343 comprises optogenetic sensitive cells, such as to biologically control the pace of the heart by triggering the cells via the LED.

In some embodiments, system 10 is configured to deliver one or more chemical agents into tissue (e.g. agent 343). Agent 343 can comprise a bulking agent (e.g. a cardiac tissue-bulking agent), and device 100 can deliver the bulking agent (e.g. via needle assembly 140) into tissue, such as heart wall tissue. For example, a bulking agent can be delivered to the wall of the left ventricle to reshape the wall to treat heart failure.

In some embodiments, system 10 is configured to excise unhealthy tissue from a patient. For example, using biopsy module 344, device 100 can remove tissue proximate a tissue surface. In some embodiments, system 10 is configured to remove a portion of unhealthy tissue, and to ablate a remaining portion of unhealthy tissue, for example, in order to perform one or more diagnostic tests on the excised portion of unhealthy tissue. In some embodiments, system 10 is configured to remove epicardial brown adipose tissue, such as to treat an arrhythmia.

Referring now to FIG. 2, a flowchart of a method of maneuvering a device to a desired tissue location is illustrated, consistent with the present inventive concepts. Method A describes inserting a device, such as device 100 described herein, into a patient, maneuvering the device to a desired tissue location, and performing a procedure at the desired location. In Step A100, device 100 is inserted into the patient and positioned proximate a tissue surface. For example, when treating cardiac tissue, device 100 can be inserted into the chest of the patient, through the pericardium, and placed onto the epicardial surface, for example, near the apex of the heart. The epicardial surface can be accessed through a minimally invasive subxiphoid incision. Once placed proximate the tissue surface, both of attachment assemblies 120a and 120b can be attached to the tissue surface to secure device 100 to the tissue surface. For example, and as described herein, suction can be applied to the tissue surface via grippers 125a and 125b to securely attach each of attachment assemblies 120a and 120b to the tissue surface.

In Step A120, system 10 (e.g. via algorithm 315) determines the location of device 100 relative to a desired treatment location and calculates a movement path along which to maneuver device 100 from the current location to the treatment location. In some embodiments, algorithm 315 determines the location of device 100 utilizing information provided by sensor interface 350. The movement path can comprise a number of footstep locations, as described herein, whereby each attachment assembly 120 is to be sequentially placed as device 100 “walks” along the movement path. In some embodiments, system 10 is configured to automatically maneuver device 100 along at least a portion of the movement path, such as by robotically or otherwise automatically advancing each attachment assembly 120, in an alternating fashion, to the next footstep location along the movement path. Alternatively or additionally, an operator of system 10 can manually rotate device 100 about a first attachment assembly 120 that is attached to tissue and acts as a pivot point, to position a second (opposite), unattached attachment assembly 120 proximate the next footstep location. In some embodiments, device 100 can be manually or semi-automatically maneuvered while the position of device 100 relative to the patient is displayed to the operator via display 410 (e.g. via fluoroscopic and/or ultrasonic imaging).

In Step A130, if device 100 is located in a position such that hub assembly 130 is sufficiently proximate a desired tissue location to perform a treatment, method A continues to Step A190 and the treatment is performed. In some embodiments, immediately following insertion (e.g. in the inserted location), a first treatment step is performed, such as a cardiac mapping procedure step, before hub assembly 130 is repositioned to the next desired tissue location. In some embodiments, before, during, and/or after each maneuver of device 100, a treatment step can be performed. If hub assembly 130 is not in the desired treatment location, or a treatment step has been performed and an additional maneuver is desired, method A continues to Step A140.

In Step A140, first attachment assembly 120a is released from the tissue (e.g. vacuum is released as described herein), and device 100 is articulated about second attachment assembly 120b to reposition first attachment assembly 120a to the next footstep location. Subsequently, in Step A150, first attachment assembly 120a is reattached to the tissue surface (e.g. vacuum is applied as described herein).

In Step A160, if device 100 is located in a position such that hub assembly 130 is proximate a desired treatment location, method A continues to Step A190 and the desired treatment is performed. Otherwise, method A continues to Step A170.

In Step A170, second attachment assembly 120b is released from the tissue, and device 100 is articulated about first attachment assembly 120a to reposition second attachment assembly 120b to the next footstep location. Subsequently, in Step A180, second attachment assembly 120b is reattached to the tissue surface. After second attachment assembly 120b has been repositioned, method A returns to Step A130. Steps A130 through A180 are repeated until hub assembly 130 is positioned proximate a desired treatment location.

Once device 100 has followed the determined movement path, and hub assembly 130 is proximate the desired treatment location, a treatment is performed in Step A190 at the desired treatment location. In Step A200, if subsequent procedures are to be performed, method A returns to either Step A140 or A170, to maneuver device 100 about second attachment assembly 120b or first attachment assembly 120a, respectively. Otherwise, if the procedure is complete, in Step A210 both attachment assemblies 120 can be released from the tissue and device 100 can be removed from the patient. In some embodiments, device 100 is maneuvered along a movement path back to the original insertion location before removal from the patient.

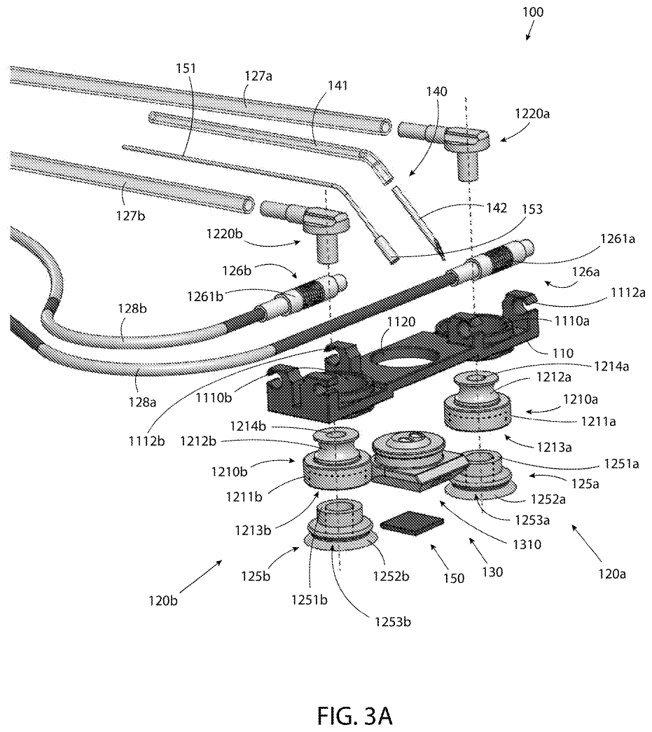

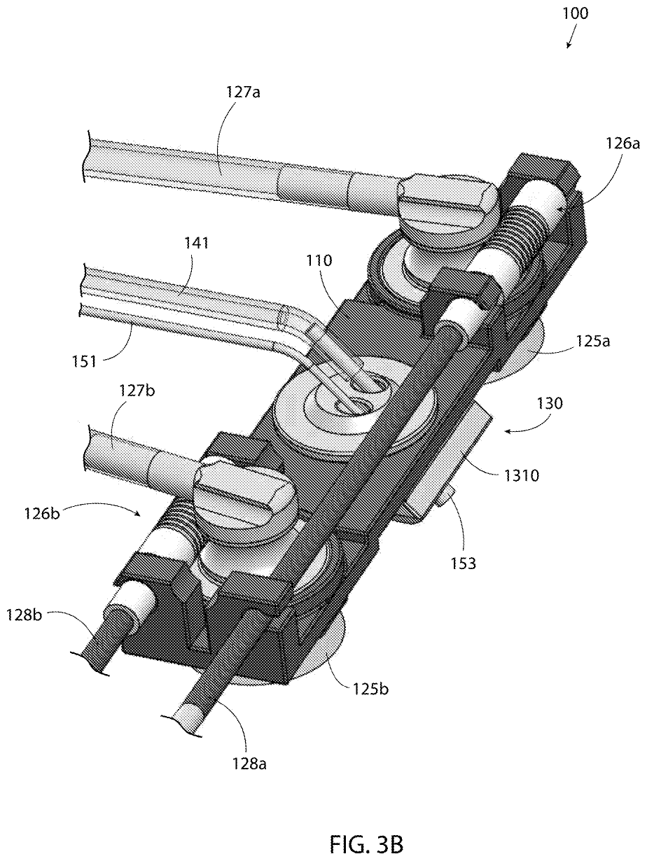

Referring now to FIGS. 3A and 3B, an exploded perspective view and a perspective view of a treatment device are illustrated, respectively, consistent with the present inventive concepts. Device 100 can be of similar construction and arrangement to device 100 described herein in reference to FIG. 1. Device 100 can comprise one, two, or more attachment assemblies, attachment assemblies 120a and 120b shown, which are described singly and/or collectively herein. Attachment assemblies 120a and 120b (singly or collectively attachment assembly 120 herein), can include similar or dissimilar components. For example, attachment assemblies 120a and 120b can each comprise a rotation control assembly 126 (e.g. assemblies 126a and 126b shown, singly or collectively rotation control assembly 126), as well as other similar components labeled in an “a” and “b” fashion. Device 100 comprises chassis 110. Chassis 110 includes two recesses, recess 1110a and recess 1110b, each constructed and arranged to rotatably receive a rotating portion of attachment assemblies 120a and 120b, hubs 1210a and 1210b, respectively. Rotation control assembly 126 (e.g. each rotation control assembly 126) is operably attached to a control conduit 128. Rotation control assembly 126 can comprise a torsional force transfer mechanism, position drive 1261, configured to operably engage hub 1210, such as to rotate hub 1210 relative to chassis 110.

Hub 1210 can include a first portion comprising a surface, bearing surface 1211, which can be constructed and arranged to slidingly engage recess 1110, such that hub 1210 can rotate within recess 1110. Hub 1210 can include a second portion, engagement portion 1212, which can be configured to operably engage with drive 1261. In some embodiments, engagement portion 1212 comprises a gear (not shown), and drive 1261 can comprise a worm screw configured to operably interact with a gear-based engagement portion 1212, such that rotation of drive 1261 about its central axis causes a perpendicular rotation of hub 1210 about its central axis, relative to chassis 110. In some embodiments, chassis 110 comprises one or more retention mechanisms, clips 1112. Clips 1112 can be constructed and arranged to attach to a portion of rotation control assembly 126 and position drive 1261, such that drive 1261 remains in contact with engagement portion 1212 of hub 1210.

Each hub 1210 can comprise a recess, recess 1213, and a lumen 1214 extending from recess 1213 through the top surface of hub 1210. Each attachment conduit 127 can include a connector, connector 1220, which can be configured to operably attach conduit 127 to hub 1210, such that the lumen of conduit 127 is fluidically connected to recess 1213 via lumen 1214.

Attachment assembly 120 can comprise gripper 125, such as is described herein in reference to FIG. 1. At least a portion of gripper 125 can be positioned within recess 1213, such as when gripper 125 is fixedly positioned within recess 1213. Gripper 125 can comprise a hollow tube defined by walls 1251. At least a portion of walls 1251 can be positioned within recess 1213, such that the inner surface of walls 1251 and recess 1213 define a chamber, suction chamber 1253. Suction chamber 1253 can be fluidically connected to the lumen of conduit 127. Gripper 125 can comprise a flexible radial projection, skirt 1252 shown, that extends from the bottom of walls 1251. Skirt 1252 can be configured to engage a tissue surface to create a seal (e.g. a relatively airtight seal) between the tissue surface and chamber 1253, such that while a vacuum is applied to chamber 1253 (e.g. via attachment conduit 127 that is attached to a source of vacuum supplied by attachment control module 320), tissue attachment assembly 120 is attached to the tissue via the vacuum pressure.

Hub assembly 130 can comprise housing 1310. Chassis 110 can include recess 1120 which can be constructed and arranged to receive at least a portion of housing 1310, such as to attach hub assembly 130 to chassis 110. In some embodiments, housing 1310 is constructed and arranged to rotate within recess 1120 relative to chassis 110. Housing 1310 can slidingly receive at least a portion of needle assembly 140 and/or ultrasound transducer 153, such as to position needle 142 and/or ultrasound transducer 153 to be proximate to tissue (e.g. within 5 mm, 3 mm, 2 mm, and/or 1 mm of a desired tissue location) and/or oriented toward tissue (e.g. within 30°, 15°. 10°. and/or 5° of a desired angular orientation). In some embodiments, electronics assembly 150 is fixedly attached to a tissue-facing portion of housing 1310, such that at least a portion of electronics assembly 150 is positioned proximate tissue (e.g. within 5 mm, 3 mm, 2 mm, and/or 1 mm of a desired tissue location) when device 100 is in contact with tissue.

Referring now to FIGS. 4A and 4B, an exploded perspective view and a perspective view of a treatment device are illustrated, respectively, consistent with the present inventive concepts. Device 100 can be of similar construction and arrangement to device 100 described herein in reference to FIG. 1. Device 100 can comprise one, two, or more attachment assemblies, such as attachment assemblies 120a and 120b shown. Attachment assemblies 120a and 120b (singly or collectively attachment assembly 120 herein), can include similar or dissimilar components. For example, attachment assemblies 120a and 120b can each comprise a gripper, such as described herein, (e.g. grippers 125a and 125b shown, singly or collectively gripper 125), as well as other similar components labeled in an “a” and “b” fashion. Device 100 comprises chassis 110. Chassis 110 can comprise an elongate body and can include a hole through a distal portion of the elongate body, hole 1111a.

Device 100 can include a component configured to pivot relative to hole 1111a, articulating arm 1240. Arm 1240 can include a projection, axle 1241, constructed and arranged to rotate within hole 1111a. In some embodiments, arm 1240 includes pulley 1242 positioned about at least a portion of axle 1241. Device 100 can include a first steering cable, cable 1282a. Device 100 can include a conduit configured to slidingly receive cable 1282a, control conduit 1281. Conduit 1281 can comprise a multi-lumen conduit, for example, a multi-lumen conduit configured to slidingly receive cable 1282a in a first lumen, and a second steering cable, cable 1282b in a second lumen. Cable 1282a can extend through conduit 1281 (e.g. from articulation control module 330 of console 300), around pulley 1242, and return through conduit 1281, such that pulling on either end of cable 1282a causes the articulation of arm 1240 about axle 1241 (e.g. relative to chassis 110). Arm 1240 can include a first projection, projection 1243a, and a second projection, projection 1243b. A lumen, lumen 1244, extends through the two projections 1243a,b. Gripper 125a can comprise a tubular structure with an end sealed with a flexible substrate, membrane 1254a. Gripper 125a can be configured to attach to projection 1243b, such that membrane 1254a forms a seal at the end of lumen 1244. Attachment conduit 127a can include a connector, connector 1220a, which can be configured to operably attach conduit 127a to lumen 1244, such that the lumen of conduit 127a is fluidically attached to a chamber formed by lumen 1244 and membrane 1254a. Gripper 125a can comprise a flexible projection, skirt 1252a, that radially extends from the tubular structure of gripper 125a. Skirt 1252a can be configured to engage a tissue surface to create a seal (e.g. a relatively airtight seal) between the tissue surface and membrane 1254a (e.g. causing tissue attachment assembly 120a to be attached to tissue, as described herein). Gripper 125a can be constructed and arranged such that while a vacuum is applied to lumen 1244 (e.g. via attachment conduit 127a that is attached to a source of vacuum supplied by attachment control module 320), membrane 1254a is drawn away from the tissue by the vacuum within lumen 1244, and a vacuum chamber is created between the tissue, skirt 1252a, and membrane 1254a, thereby attaching tissue attachment assembly 120a to the tissue via the vacuum pressure. In some embodiments, conduit 127a includes a service loop (e.g. a serpentine service loop as shown) that is constructed and arranged to allow arm 1240 to articulate relative to chassis 110 without impediment from conduit 127a. In some embodiments, chassis 110 comprises a relief, slot 1113. Slot 1113 can provide clearance for connector 1220a while arm 1240 rotates about axle 1241 (e.g. such that chassis 110 does not interfere with the rotation by blocking connector 1220a).

Chassis 110 can comprise a hole, hole 1111b, extending through both sides of a mid-portion of the elongate body of chassis 110. Device 100 can include an assembly configured to pivot relative to hole 1111b, articulating assembly 1400. Assembly 1400 can comprise a first component, structure 1401a that includes hollow projection 1403, and a second component, structure 1401b that includes recess 1404. Structure 1401a comprises a first portion 1431 that extends proximally from projection 1403 along a first side of chassis 110, a second portion 1432, which partially surrounds a portion of chassis 110, and a third portion 1433 that aligns with structure 1401b on a second side of chassis 110. Assembly 1400 can include an axle 1406, and a pulley 1405 that is configured to slidingly receive axle 1406. Axle 1406 can be positioned with a first end inserted into hollow projection 1403. Axle 1406 can extend through a first portion of hole 1111b, through pulley 1405, through a second portion of hole 1111b (e.g. when pulley 1405 is positioned within a cavity of chassis 110), and into recess 1404. In some embodiments axle 1406 comprises a keyed shape, such that the axial orientations of first structure 1401a, pulley 1405, and second structure 1401b are fixed relative to each other via axle 1406. Device 100 can include a second steering cable, cable 1282b. Control conduit 1281 can be configured to slidingly receive cable 1282b. Cable 1282b can extend through conduit 1281 (e.g. from articulation control module 330 of console 300), around pulley 1405, and return through conduit 1281, such that pulling on either end of cable 1282b causes the articulation of assembly 1400 about axle 1406 (e.g. relative to chassis 110).

Articulating assembly 1400 can comprise a translatable component, cart 1409. Cart 1409 can be configured to translate linearly along an elongate member, rail 1407, positioned between third portion 1433 of structure 1401a and second structure 1401b (e.g. such that cart 1409 can translate between third portion 1433 and second structure 1401b). Assembly 1400 can comprise a biasing member, spring 1408, positioned between cart 1409 and second structure 1401b, such as to bias cart 1409 towards third portion 1433 of structure 1401a.

Device 100 can include needle assembly 140 which can comprise needle 142 that is fluidically attached to conduit 141. At least a portion of needle assembly 140 can be slidingly received through a lumen extending through third portion 1433, fixedly positioned within a lumen of cart 1409, and extend slidingly through a lumen of structure 1401b. At least a portion of needle assembly 140 can be attached to cart 1409, such that when cart 1409 translates, at least a portion of needle 142 translates in unison with cart 1409 (e.g. relative to third portion 1433 and extending out of second structure 1401b). In some embodiments, conduit 141 is constructed and arranged to control the translation of cart 1409 and needle 142 (e.g. when conduit 141 is advanced via a force received from console 300, cart 1409 and needle 142 advance in response). In some embodiments, needle assembly 140 comprises conduit 1411, which can be fixedly attached to third portion 1433 and configured to slidingly receive needle 142 and/or conduit 141.

Device 100 can include ultrasound transducer 153, which can be operably attached to communication conduit 151. Ultrasound transducer 153 can be attached to structure 1401b, for example, such that the functional end of ultrasound transducer 153 is positioned proximate the distal end of structure 1401b. Device 100 can comprise one or more electronics assemblies, such as assemblies 150a and 150b shown. Electronics assembly 150a can be attached to the underside (e.g. as shown on the page) of chassis 110, and electronics assembly 150b can be attached to the top side of chassis 110.

Chassis 110 can comprise a projection 1114, and a lumen 1115 that extends from the proximal end of chassis 110 through projection 1114. Gripper 125b can be of similar construction and arrangement to gripper 125a, and can be configured to attach to projection 1114, such that membrane 1254b forms a seal at the end of lumen 1115. Attachment conduit 127b can be configured to operably attach to lumen 1115, such that the lumen of conduit 127b is fluidically attached to a chamber formed by lumen 1115 and membrane 1254b. Gripper 125b can comprise a flexible projection, skirt 1252b, that radially extends from the tubular structure of gripper 125b. Skirt 1252b can be configured to engage a tissue surface to create a seal (e.g. a relatively airtight seal) between the tissue surface and membrane 1254b (e.g. causing tissue attachment assembly 120b to be attached to tissue, as described herein). Gripper 125b can be constructed and arranged such that while a vacuum is applied to lumen 1115 (e.g. via attachment conduit 127b that is attached to a source of vacuum supplied by attachment control module 320), membrane 1254b is drawn away from the tissue by the vacuum within lumen 1115, and a vacuum chamber is created between the tissue, skirt 1252b, and membrane 1254b, and tissue attachment assembly 120b is attached to the tissue via the vacuum pressure.

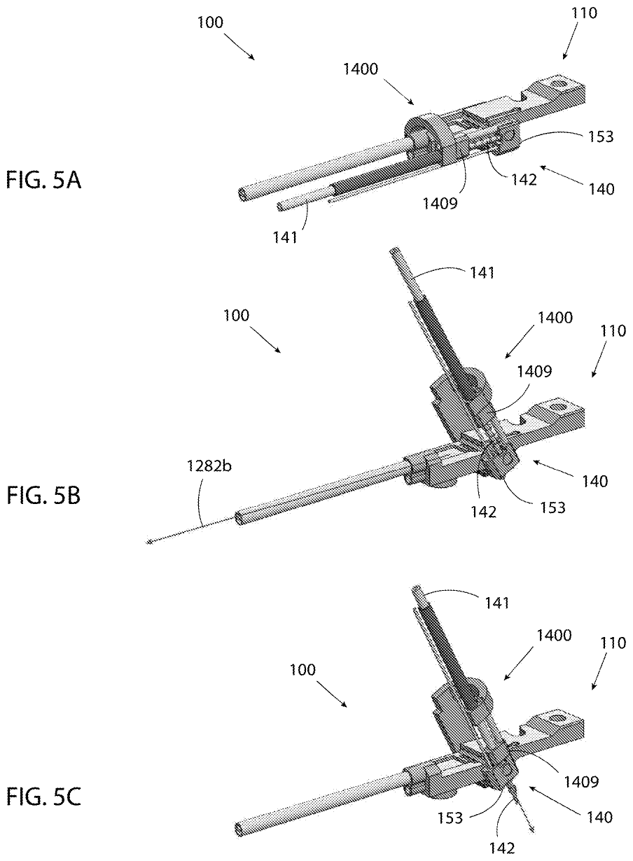

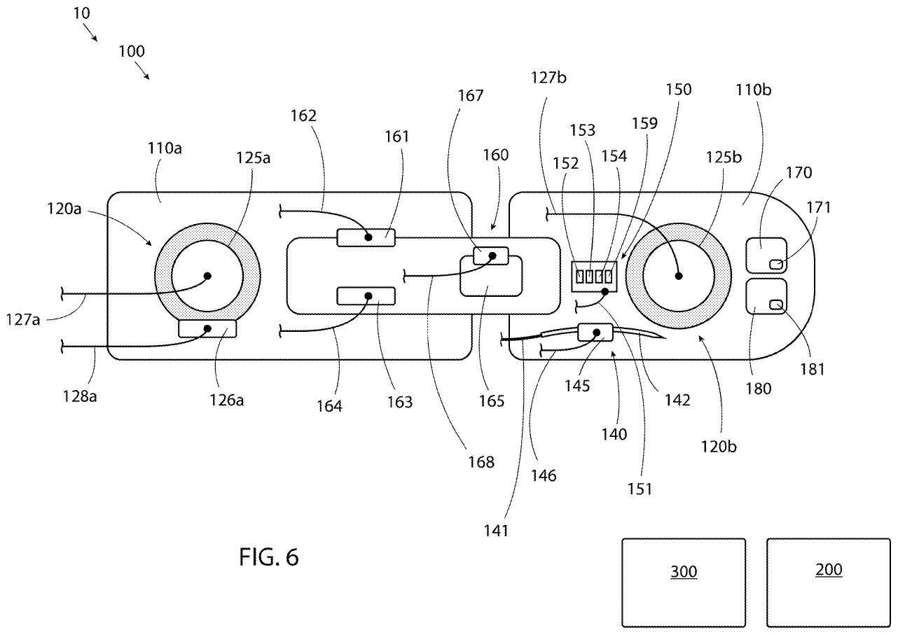

Referring now to FIGS. 5A-C, perspective views of a treatment device in various deployment positions are illustrated, consistent with the present inventive concepts. Device 100 illustrated in FIGS. 5A-5C can be of similar construction and arrangement and comprise similar components to device 100 described in FIGS. 4A and 4B herein. In FIGS. 5A-C, some components of device 100 have been removed for illustrative clarity. Articulating assembly 1400 and needle assembly 140 are shown in various orientations relative to chassis 110. In FIG. 5A, articulating assembly 1400 is illustrated in a lowered position, relatively in line with chassis 110, such that device 100 comprises a relatively streamlined overall shape (e.g. for ease of insertion and/or navigation within a patient). In FIG. 5B, articulation assembly 1400 has been articulated (e.g. advanced at least 1 mm, 2 mm, 3 mm, and/or 5 mm and/or rotated through an arc of at least 5°, 15°, and/or 30°) into a raised position, such that needle assembly 140 and ultrasound transducer 153 are oriented relatively towards tissue (e.g. oriented towards the underside of chassis 110 as shown on the page). In some embodiments, steering cable 1282b is manipulated to control the articulation of assembly 1400 (e.g. one end of cable 1282b has been pulled to articulate assembly 1400 from the orientation illustrated in FIG. 5A to the orientation illustrated in FIG. 5B). In FIG. 5C, needle 142 (and cart 1409) have been advanced (e.g. advanced at least 0.5 mm, 1.0 mm, and/or 1.5 mm), for example, such that needle 142 is advanced into tissue. In some embodiments, conduit 141 is advanced to control the advancement of needle 142 (e.g. conduit 141 has been advanced to advance needle 142 to the position illustrated in FIG. 5C). Referring now to FIG. 6, a schematic view of a system including a device for diagnosing and/or treating a patient is illustrated, consistent with the present inventive concepts. Device 100, interface assembly 200, and console 300 of system 10 can be of similar construction and arrangement to device 100, interface assembly 200, and console 300 described in reference to FIG. 1 and otherwise herein. Device 100 comprises a mobile device that can be constructed and arranged to operably attach to console 300 via interface assembly 200 as shown.

In some embodiments, device 100 comprises a two-part chassis, including base chassis 110a and nose chassis 110b as illustrated. Base chassis 110a can be operably attached to nose chassis 110b via connection assembly 160. Connection assembly 160 can be constructed and arranged to connect base chassis 110a and nose chassis 110b with one, two, three, or more degrees of freedom. For example, nose chassis 110b can be articulated relative to base chassis 110a in an arrangement selected from the group consisting of: longitudinally along a longitudinal axis of device 100 (e.g. “away from” or “towards” base chassis 110a, such as an advancement of at least 1 mm, 2 mm, 3 mm, and/or 5 mm); rotationally about the pitch axis of device 100, or an axis parallel to a horizontal plane of device 100 (e.g. rotated “up” and/or “down” relative to base chassis 110a, such as a rotation through an arc of at least 5°, 15°, and/or 30°); rotationally about the yaw axis of device 100, or an axis perpendicular to a horizontal plane of device 100 (e.g. rotates “left” and/or “right” relative to base chassis 110a, such as a rotation through an arc of at least 5°, 15°, and/or 30°); rotationally about the roll axis of device 100, or a longitudinal axis of device 100 (e.g. rolled clockwise and/or counter-clockwise relative to base chassis 110a, such as a rotation through an arc of at least 5°, 15°, and/or 30°); and combinations of these.

Device 100 can include one or more assemblies for controlling the articulation of nose chassis 110b relative to base chassis 110a. For example, device 100 can include translation assembly 161 which can be configured to control the longitudinal extension of nose chassis 110b relative to base chassis 110a (e.g. an advancement of at least 1 mm, 2 mm, 3 mm, and/or 5 mm). Translation control assembly 161 can be operably attached to console 300 of system 10 via a conduit, control conduit 162. Additionally or alternatively, device 100 can include rotation control assembly 163 which can be configured to control the rotational position of nose chassis 110b relative to base chassis 110a (e.g. the yaw of nose chassis 110b, such as a rotation through an arc of at least 5°, 15°, and/or 30°). Rotation control assembly 163 can be operably attached to console 300 of system 10 via a conduit, control conduit 164. Additionally or alternatively, device 100 can include articulation control assembly 167 which can be configured to control the articulation of nose chassis 110b relative to base chassis 110a (e.g. the pitch of nose chassis 110b, such as a rotation through an arc of at least 5°, 15°, and/or 30°). Articulation control assembly 167 can be operably attached to console 300 of system 10 via a conduit, control conduit 168. Assemblies 161, 163, and/or 167 can operably connect (e.g. via conduits 162, 164, and/or 168, respectively) to articulation control module 330 of console 300 of system 10 (not shown in FIG. 6 but such as is described herein).

In some embodiments, connection assembly 160 comprises one or more hinges, hinge 165 shown. Hinge 165 can comprise a hinge providing one, two, three, or more degrees of freedom between base chassis 110a and nose chassis 110b. In some embodiments, hinge 165 comprises a universal joint, providing two or more degrees of freedom. In some embodiments, hinge 165 comprises two or more portions constructed and arranged to articulate in the same direction at different points along connection assembly 160. For example, hinge 165 can be constructed and arranged to allow nose chassis 110b to pitch about base chassis 110a, such as a pitch about two or more articulation points. In some embodiments, hinge 165 can comprise a living hinge providing one, two, three, or more degrees of freedom.

Device 100 can include a first tissue attachment assembly, base tissue attachment assembly 120a, which is operably attached to base chassis 110a. Base tissue attachment assembly 120a can be constructed and arranged to cause base chassis 110a to securely attach to (e.g. engage with) and/or detach from (e.g. disengage with) tissue (e.g. a tissue surface), for example as described herein. Base tissue attachment assembly 120a can comprise at least one mechanism for the tissue attachment and detachment, gripper 125a shown. In some embodiments base tissue attachment assembly 120a further comprises a conduit, attachment conduit 127a, that is operably connected to console 300 as described herein. Base tissue attachment assembly 120a can comprise rotational control assembly 126a which is operably attached to control conduit 128a and configured to rotate base chassis 110a relative to base tissue attachment assembly 120a.

Device 100 can include a second tissue attachment assembly, nose tissue attachment assembly 120b, which is operably attached to nose chassis 110b. Nose tissue attachment assembly 120b can be constructed and arranged to operably attach and/or detach nose chassis 110b to and/or from tissue, for example as described herein. Nose tissue attachment assembly 120b can comprise at least one mechanism for the tissue attachment and detachment, gripper 125b shown. In some embodiments nose tissue attachment assembly 120b further comprises a conduit, attachment conduit 127b, that is operably connected to console 300 as described herein.

Device 100 can include one or more electronic modules, electronics assembly 150 shown. Electronics assembly 150 can be of similar construction and arrangement to electronics assembly 150 described in reference to FIG. 1 and otherwise herein. Electronics assembly 150 can include sensor 152, ultrasound transducer 153, electrode 154, functional element 159, and/or other electronic elements, such as are described herein. In some embodiments, one or more portions of electronics assembly 150 is positioned on nose chassis 110b and/or on base chassis 110a. Electronics assembly 150 can be operably attached to communication conduit 151.

Device 100 can include one or more assemblies configured to deliver one or more needles or other filaments into tissue, needle assembly 140 shown. Needle assembly 140 can include one or more needles or other filaments, needle 142 shown, which can comprise a needle or other filament operably (e.g. fluidically or electrically) connected to conduit 141. Conduit 141 can operably attach needle 142 to console 300, as described herein. Needle assembly 140 can include needle articulation control assembly 145. Control assembly 145 can be constructed and arranged to advance needle 142 away from a surface of device 100, for example from nose chassis 110b of device 100 and into tissue (e.g. an advancement into tissue of at least 1 mm, 2 mm, 3 mm, and/or 5 mm). Control assembly 145 can be operably attached to a conduit, control conduit 146. Control conduit 146 can be operably attached to console 300, such that console 300 can provide control of the actuation of needle 142 via control assembly 145.

In some embodiments, one or more control assemblies of device 100 comprise an assembly that is adjusted via a rotation of a portion of that assembly. For example, the assembly can include a worm gear, lead screw and nut, or other rotationally driven mechanism configured to translate and/or otherwise manipulate a second portion of the assembly when a first portion is rotated. Various embodiments of such assemblies are described herein. In some embodiments, one or more control conduits (e.g. control conduits operably attached to the control assemblies) comprise torque cables constructed and arranged to transfer torsional force to the control assemblies (e.g. from console 300).

In some embodiments, device 100 further comprises one or more tissue sensing modules, TSM 170 shown, and/or device 100 can further comprise one or more tissue dissection modules, TDM 180 shown. In some embodiments, nose chassis 110b and/or tissue attachment assembly 120b can comprise TSM 170 and/or TDM 180. TSM 170 can include one or more sensors, sensor 171 shown. The sensors 171 can include impedance electrodes, ultrasound transducers, strain gages, and/or imaging elements (e.g. video camera components). In some embodiments, the sensors 171 can be small (for example having a minor or major axis that is no more than 1 mm or 2 mm). Device 100 can include built-in serial and/or bus-based communications capabilities for signals produced by sensors 171. Sensors 171 can comprise ultrasound transducers that are configured as proximity sensors. Sensors 171 can comprise strain gages that include a flexible chamber in fluid connection with a pressure gage. Sensors 171 can comprise a visualization device (e.g. a video camera) such as a polychrome or monochrome device, and the visualization device can comprise: a field view of at least 45°, 90°, 120°, or 170°; a variable focal depth or a fixed focal depth of 2-20 mm; and/or a light source. Sensors 171 can comprise a visualization device with a light source comprising a local LED and/or an optical fiber connected to a narrow or broad band illumination source.

Sensors 171 can be positioned in the distal portion of nose chassis 110b and/or in a distal portion of tissue attachment assembly 120b. For example, sensors 171 can be positioned to contact tissue that lies ±30° or ±45° or ±90° off the distal end of the device (e.g. off a central axis of the distal end of the device). Sensors 171 can be configured to detect the presence of tissue, locate tissue (e.g. relative to a portion of device 100), and/or characterize tissue that may obstruct the intended navigation path of device 100. For example, such tissue can constitute a pericardial adhesion. Multiple sensors 171 can be included to reduce the movement of device 100 that is required to obtain sufficient data to produce a map of potential obstructing tissue ahead of the intended path of device 100.

TDM 180 can comprise one or more tools, tool 181 shown, such as a tool for dissecting tissue. For example, tool 181 can comprise electrocautery and/or ultrasonic cutting tools. An electrocautery tool can comprise a monopolar or bipolar cautery surface (e.g. a bipolar tool configured to confine the cutting energy to the area that spans TDM 180). Tool 181 can comprise electrodes that are part of sensors 171, for example electrodes that are also configured to deliver energy, such as to cut tissue.

Tool 181 can comprise a cutting tool that is positioned on (e.g. wrapped around) the distal end of the device 100 so that its cutting surface will span the front of the device 100 and wrap at least partially around nose chassis 110b such that nose chassis 110b can be either pushed directly into an adhesion and/or rotated to the side to contact an adhesion.

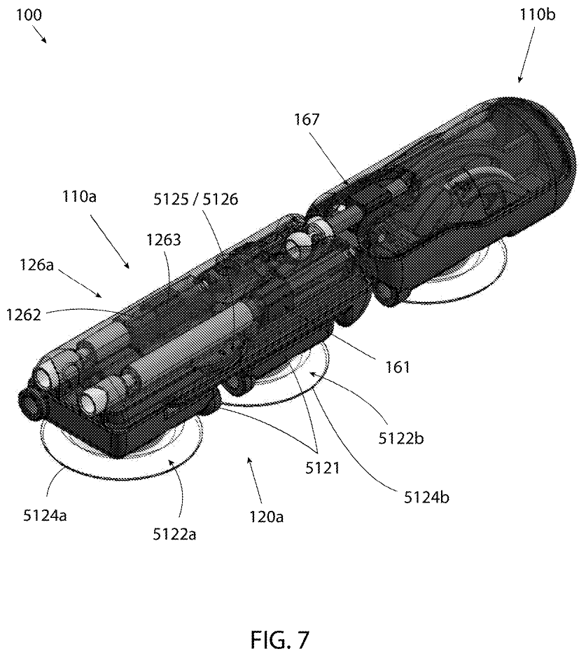

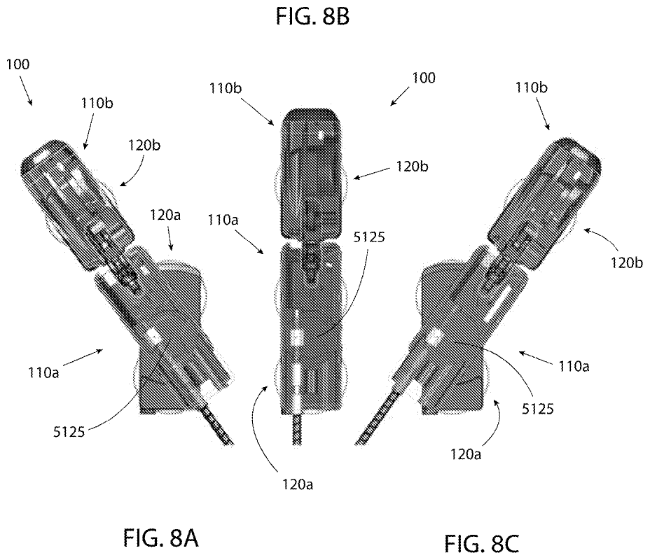

Referring now to FIG. 7, a perspective view of a treatment device is illustrated, consistent with the present inventive concepts. Device 100 can be of similar construction and arrangement to device 100 described herein. Device 100 can comprise base chassis 110a and nose chassis 110b. Base chassis 110a can include base tissue attachment assembly 120a. In some embodiments, base tissue attachment assembly 120a comprises a rotatable structure, housing 5121. Attachment assembly 120a can comprise two attachment assemblies, 5122a and 5122b, positioned at opposite ends of housing 5121 as shown. Housing 5121 can comprise two chambers, 5123a,b, not shown, but configured to operably attach to respective grippers 5124a,b. Housing 5121 can comprise a projection, hub 5125, configured to be rotatingly received within a recess of base chassis 110a, recess 5126. Rotation control assembly 126a can comprise a drive mechanism, lead screw 1262, operably engaged with a threaded projection extending from hub 5125, nut 1263. Lead screw 1262 and nut 1263 can be constructed and arranged such that as lead screw 1262 is rotated, nut 1263 translates along lead screw 1262 and housing 5121 rotates about hub 5125. During use (e.g. use during a clinical procedure), attachment assembly 120a can be attached to tissue (e.g. attachment assemblies 5122a,b are both attached to tissue), and device 100 can be rotated about hub 5125 relative to the tissue. For example, device 100 can be rotated to adjust the position of nose chassis 110b relative to attachment assembly 120a as illustrated in FIGS. 8A-C, described herein.

Referring additionally to FIGS. 8A-C, three articulated orientations of device 100 are illustrated, consistent with the present inventive concepts. In FIG. 8A, nose chassis 110b is illustrated articulated to the left (on the page), as device 100 has been rotated counterclockwise about hub 5125. In FIG. 8B, nose chassis 110b is illustrated in line with base chassis 110a. In FIG. 8C, nose chassis 110b is illustrated articulated to the right, as device 100 has been rotated clockwise about hub 5125. During use, after nose chassis 110b, including attachment assembly 120b, has been articulated relative to attachment assembly 120a (e.g. while attachment assembly 120a is attached to tissue), attachment assembly 120b can then be attached to tissue, as described herein. After attachment assembly 120b is attached to tissue, attachment assembly 120a can be detached from tissue, and housing 5121 can be rotated to realign with nose chassis 110b (e.g. to align with nose chassis 110b in its new orientation).

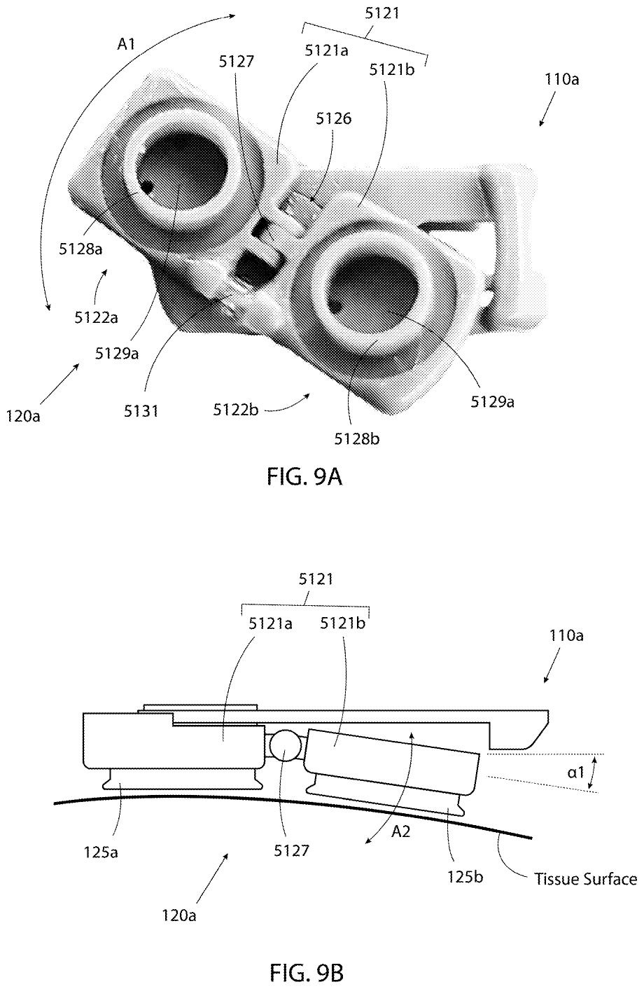

Referring additionally to FIGS. 9A and 9B, a bottom and a side view of a portion of an attachment assembly are illustrated, respectively, consistent with the present inventive concepts. In FIG. 9A, base tissue attachment assembly 120a is illustrated from the underside, in an orientation in which attachment assembly 120a rotated relative to a portion of base chassis 110a. In some embodiments, housing 5121 comprises a first housing 5121a, and a second housing 5121b. First housing 5121a can provide at least a portion of tissue attachment assembly 5122a, and second housing 5121b can provide at least a portion of tissue attachment assembly 5122b (e.g. such that assemblies 5122a,b rotate and/or otherwise articulate with the respective housings 5121a,b). Housings 5121a,b can be attached via an articulatable attachment mechanism, hinge 5127. Hinge 5127 can be constructed and arranged such that housings 5121a,b rotate together about recess 5126 of base chassis 110a (e.g. as indicated by arrow A1). Hinge 5127 can be constructed and arranged to allow second housing 5121b to articulate about an axis perpendicular to the axis of rotation about recess 5126, (e.g. as indicated by arrow A2 of FIG. 9B). In some embodiments, hinge 5127 comprises a living hinge, for example a living hinge that is biased such that housing 5121b is biased towards tissue (e.g. away from base chassis 110a). In FIG. 9B, second housing 5121b is illustrated in an articulated position, rotated downward, away from base chassis 110a. The articulation of housing 5121b relative to housing 5121a can provide enhanced apposition of attachment assemblies 5122a,b to tissue, for example when the tissue comprises a curved surface, as illustrated in FIG. 9B. Housing 5121b is illustrated having been rotated an angle α1 from parallel with base chassis 110a. In some embodiments, hinge 5127 allows angle α1 to be at least 5°, 10°, 15, and/or 30°. In some embodiments, hinge 5127 is biased such that housing 5121b is parallel to base chassis 110a (e.g. α1 equal to 0°) in its rest position (e.g. biased toward a level of parallelism within 20″, 10°, and/or 5°, Alternatively or additionally, binge 5127 can be biased such that housing 5121b rests at its maximum articulated position (e.g., the maximum angle α1). In some embodiments, hinge 5127 can be constructed and arranged to provide minimal resistance to the positioning of housing 5121b relative to base chassis 110a, such that housing 5121b can freely articulate to contact an uneven (e.g. curved) tissue surface beneath attachment assembly 120a when attachment assemblies 5122a,b are attached to tissue.

Housings 5121a,b can each comprise a projection, projections 5128a,b shown, that extend from a hollow interior portion of housings 5121a,b, chambers 5129a,b. Chambers 5129a,b can be of similar construction and arrangement to the various suction chambers described herein, such as chambers configured to attach tissue attachment assemblies 5122a,b to tissue when a vacuum is applied to the chamber (e.g. via a conduit 127, not shown but described herein). In some embodiments, tissue attachment assemblies 5122a,b each include a gripper 125, such as grippers 125a,b shown in FIG. 9B, which are attached to projections 5128a,b, respectively, and configured to provide a seal between chambers 5129a,b and tissue when vacuum is applied to chambers 5129a,b. In FIG. 9A, grippers 125a,b have been removed for illustrative clarity. In some embodiments, chambers 5129a,b are fluidically attached, such that a single source of vacuum can be applied to both chambers simultaneously. For example, in some embodiments chambers 5129a,b are attached via a flexible conduit, tube 5131. In some embodiments, tube 5131 comprises a material with sufficient rigidity to withstand vacuum pressure applied to chambers 5129a,b while still allowing articulation of housing 5121b relative to housing 5121a.

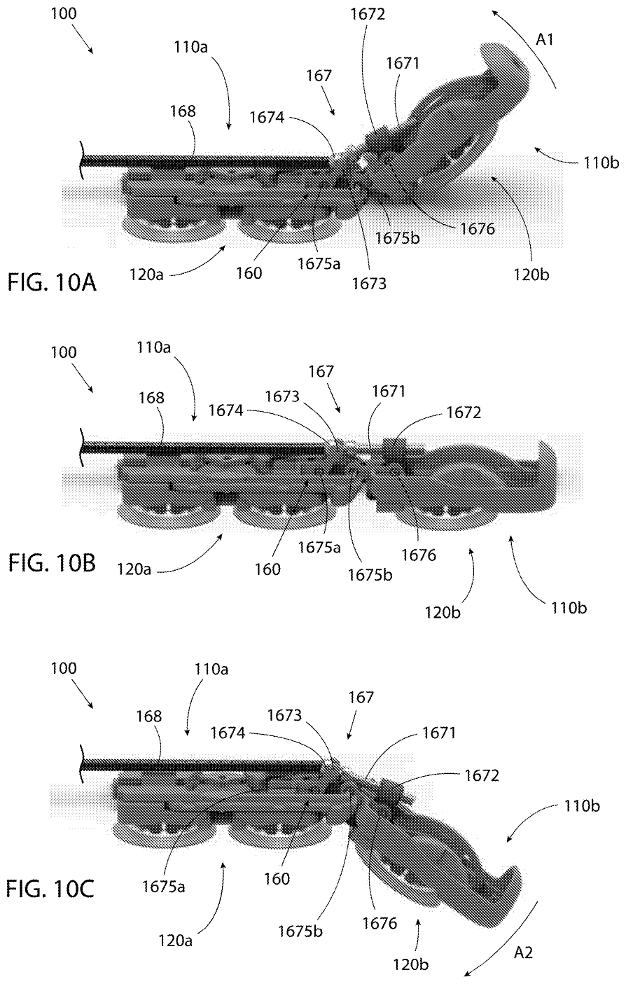

Referring now to FIGS. 10A-C, three articulated orientations of device 100 are illustrated, consistent with the present inventive concepts. In FIG. 10B, nose chassis 110b is illustrated in a neutral position, in line with base chassis 110a. In FIGS. 10A and 10C, nose chassis 110b is shown articulated (pitched) up and down, respectively, relative to base chassis 110a (as indicated by arrows A1 and A2, respectively). During use, nose chassis 110b may be controlled (e.g. by an operator and/or automatically by system 10) to pitch up and/or down to follow a tissue surface, for example a concave and/or convex tissue surface, respectively. In FIGS. 10A-C, top portions of chassis 110a,b, as well as various other components of device 100, have been removed for illustrative clarity.

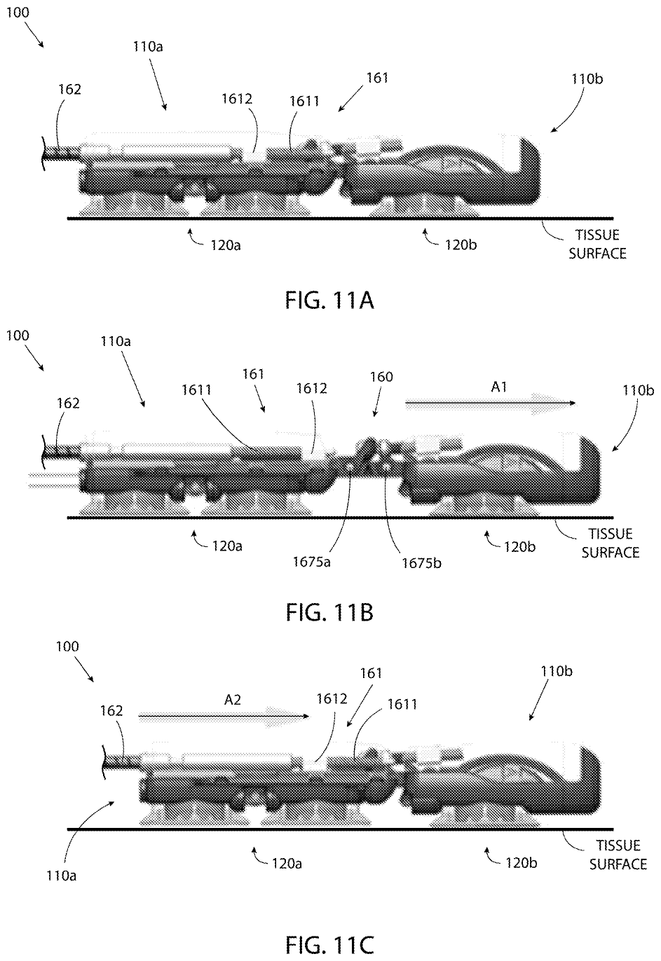

Referring additionally to FIGS. 11A-C, three configurations of device 100 illustrating forward locomotion of the device are illustrated, consistent with the present inventive concepts. In FIG. 11A, base chassis 110a and nose chassis 110b are illustrated in a first orientation, with nose chassis 110b in a retracted position relative to base chassis 110a. In FIG. 11B, nose chassis 110b is shown in an extended orientation relative to base chassis 110a. During use, nose chassis 110b may transition to this extended orientation while base tissue attachment assembly 120a is attached to tissue, such that nose chassis 110b translates forward relative to the tissue (as indicated by arrow A1). In FIG. 11C, base chassis 110a is shown in an advanced orientation (e.g. advanced to nose chassis 110b, such that device 100 is again in the orientation illustrated in FIG. 11A, but in a new location relative to the tissue). Again, during use, base chassis 110a may transition to this advanced orientation while nose tissue attachment assembly 120b is attached to tissue, such that base chassis 110a translates forward relative to the tissue (as indicated by arrow A2).

In some embodiments, articulation control assembly 167 comprises a drive mechanism, lead screw 1671, which is operably engaged with a threaded component, nut 1672. Nut 1672 can be attached to nose chassis 110b, such that as nut 1672 translates along lead screw 1671, nose chassis 110b articulates relative to base chassis 110a as illustrated. In some embodiments, nut 1672 is rotatably attached to nose chassis 110b (e.g. via an articulating joint, hinge 1676) such that as nose chassis 110b is articulated relative to base chassis 110a, nut 1672 remains aligned with lead screw 1671. In some embodiments, lead screw 1671 attaches to a portion of connection assembly 160 via an articulating joint. For example, lead screw 1671 can comprise a rounded proximal end, ball 1674, and connection assembly 160 can comprise a projection, socket 1673, configured to receive ball 1674 of lead screw 1671. Control conduit 168 can attach to lead screw 1671, such that rotation of conduit 168 in turn causes the rotation of lead screw 1671. In some embodiments, ball 1674 and socket 1673 are constructed and arranged such that ball 1674 can rotate freely about the axis of lead screw 1671 within socket 1673.

In some embodiments, connection assembly 160 comprises multiple articulation points, for example hinges 1675a,b shown. In some embodiments, connection assembly 160 comprises one or more flexible portions, such that connection assembly 160 comprises a living hinge providing one, two, or more degrees of freedom about one, two, or more axis of rotation. Articulation control assembly 167 can manipulate the articulation of nose chassis 110b about one or both of hinges 1675. For example, when in a retracted position (illustrated in FIGS. 10A-C), nose chassis 110b articulates about hinge 1675b, as hinge 1675a is constrained within base chassis 110a. Additionally, when in an advanced position (illustrated in FIG. 11B), nose chassis 110b articulates about both hinge 1675a and 1675b, as hinge 1675 has been advanced from base chassis 110a and is no longer constrained. In some embodiments, device 100 is constructed and arranged such that nose chassis 110b can articulate at least 45° up, and/or at least 45° down from the neutral position illustrated in FIG. 10B.

In some embodiments, translation control assembly 161 comprises a drive mechanism, lead screw 1611, which can be operably engaged with a threaded component, nut 1612. Nut 1612 can be attached to a portion of connection assembly 160, such that as nut 1612 translates along lead screw 1611, connection assembly 160 translates axially relative to base chassis 110a. Connection assembly 160 can be attached to nose chassis 110b, such that as connection assembly 160 translates, nose chassis 110b also translates. Control conduit 162 can attach to lead screw 1611, such that rotation of conduit 162 in turn causes the rotation of lead screw 1611. In some embodiments, device 100 is constructed and arranged such that nose chassis 110b can extend at least 2 mm, 5 mm, and/or 10 mm from base chassis 110a.

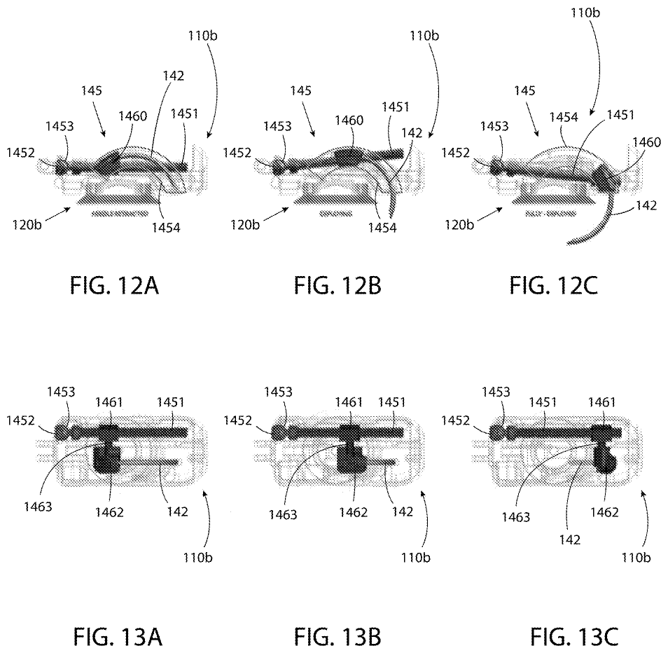

Referring now to FIGS. 12A-C and 13A-C, three side and three top views, respectively, of a needle deployment assembly are illustrated, consistent with the present inventive concepts. In FIGS. 12A and 13A, needle 142 is illustrated in a fully retracted position. In FIGS. 12B and 13B, needle 142 is illustrated in a partially deployed position. In FIGS. 12C and 13C, needle 142 is illustrated in a fully deployed position. Needle articulation control assembly 145 can be positioned within nose chassis 110b of device 100. Control assembly 145 can include one or more drive mechanisms, such as lead screw 1451 shown. Needle 142 can extend from hub assembly 1460, and assembly 1460 can translate (e.g. either direction) along lead screw 1451 such as to deploy and/or retract needle 142 from nose chassis 110b. Hub assembly 1460 can include carriage 1462. Needle 142 can extend from carriage 1462 in a curved trajectory, as shown, such that as carriage 1462 is translated along an arc, needle 142 extends along a similar path from nose chassis 110b. Carriage 1462 can be rotatably attached to a threaded component, nut 1461, via hinge 1463. Nut 1461 can be configured to operably engage lead screw 1451, such that nut 1461 translates along lead screw 1451 as screw 1451 rotates. Nose chassis 110b can comprise a groove or other recess, channel 1454, configured to slidingly receive at least a portion of hub assembly 1460. Channel 1454 can comprise a profile configured to match the shape of needle 142, such that hub assembly 1460 follows the shape of needle 142 through nose chassis 110b as needle 142 is deployed from chassis 110b. In some embodiments, the proximal end of lead screw 1451 attaches to nose chassis 110b via an articulating joint. For example, and as shown, lead screw 1451 can comprise a rounded proximal end, ball 1452, and nose chassis 110b can comprise a projection, socket 1453, where socket 1453 is configured to receive ball 1452 of lead screw 1451. A control conduit (e.g. control conduit 141 not shown but described herein) can attach to lead screw 1451, such that rotation of the conduit in turn causes the rotation of lead screw 1451. In some embodiments, ball 1452 and socket 1453 are constructed and arranged such that ball 1452 can rotate freely about the axis of lead screw 1451 within socket 1453.

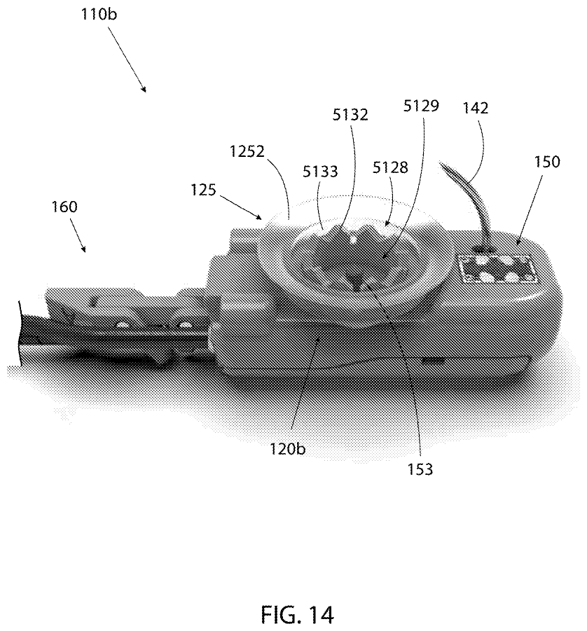

Referring now to FIG. 14, a perspective view of the underside of the nose chassis of a treatment device is illustrated, consistent with the present inventive concepts. Nose chassis 110b can be of similar construction and arrangement, and include similar components, as nose chassis 110b described in reference to FIG. 6 and otherwise herein. Nose chassis 110b includes nose tissue attachment assembly 120b. A connection assembly, assembly 160 extends proximally from nose chassis 110b. In some embodiments, electronics assembly 150 is positioned on a distal portion of nose chassis 110b, for example distal to attachment assembly 120b. In some embodiments, ultrasound transducer 153 is positioned within chamber 5129 of attachment assembly 120b, as shown. In some embodiments, at least a portion of electronics assembly 150 is positioned within chamber 5129. For example, at least a portion of electronics assembly 150 can be positioned to surround at least a portion of transducer 153 within chamber 5129 (e.g. assembly 150 includes a hole, and a distal end of transducer 153 is positioned through or otherwise proximate the hole). In some embodiments, tissue attachment assembly 120b is configured to attach to tissue and to ensure adequate contact (e.g. to maintain a retention force with tissue of at least 1 g, 3 g, and/or 5 g) between tissue and either or both transducer 153 or electronics assembly 150 (e.g. when assembly 150 and/or transducer 153 is positioned within chamber 5129). For example, tissue attachment assembly 120b can be configured to limit relative movement between tissue and either or both of transducer 153 and electronics assembly 150 (e.g. when device 100 is positioned on a beating heart, such as to limit relative movement to at most 2 mm, 1 mm, and/or 0.5 mm). Needle 142 is illustrated in FIG. 14 in a deployed orientation, extending along a curved path from distal to attachment assembly 120b towards attachment assembly 120b.

As described herein, tissue attachment assembly 120b can comprise projection 5128 extending from nose chassis 110b towards tissue. Gripper 125 can be attached to projection 5128, such that skirt 1252 extends radially away from projection 5128 and towards tissue. Gripper 125 can be constructed and arranged to form a seal between the tissue and chamber 5129, for example when a source of vacuum is applied to chamber 5129 (e.g. to attach tissue attachment assembly 120b to tissue). In some embodiments, projection 5128 comprises a castellated profile, such as a profile including multiple peaks 5132 each separated by a valley 5133, as shown. In some embodiments, each valley 5133 comprises a major axis with a length of at least 0.1 mm, 0.3 mm, and/or 0.5 mm. In some embodiments, the profile of projection 5128 can be configured to provide mechanical interference between tissue attachment assembly 120b and tissue (e.g. when vacuum is applied to chamber 5129). In some embodiments, the profile is configured to minimize localized tissue injury caused by attaching tissue attachment assembly 120 to tissue (e.g. to prevent or minimize over compression of tissue that would cause reduced capillary circulation). Any tissue attachment assembly of device 100 can include the features of tissue attachment assembly 120b described in reference to FIG. 14.

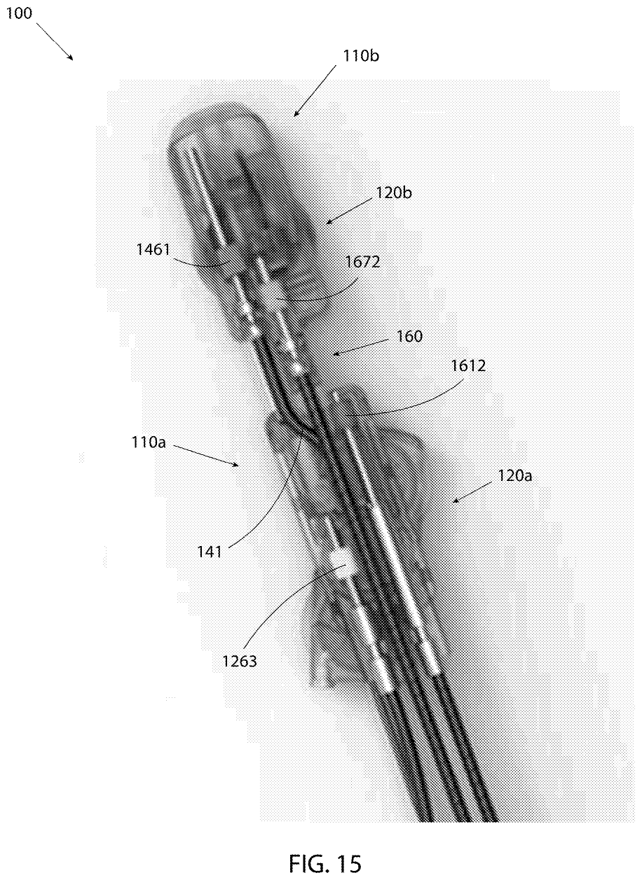

Referring now to FIG. 15, a top view of a treatment device is illustrated, consistent with the present inventive concepts. Device 100 of FIG. 15 includes nose chassis 110b which extends from base chassis 110a. Connection assembly 160 operably connects chassis 110a to chassis 110b, such as in a manner as described herein. Device 100 comprises two tissue attachment assemblies as shown, assembly 120a and assembly 120b of base chassis 110a and nose chassis 110b, respectively.

In some embodiments, base chassis 110a includes one or more recesses through which one or more control conduits of device 100 are routed (e.g. from the back of base chassis 110a to a mating portion of a lead screw). For example, control conduit 141 can be routed through base chassis 110a as shown. In some embodiments, the route of one or more conduits through base chassis 110a comprises a non-straight path, such as the path of conduit 141 shown, that includes a jog from the central axis of base chassis 110a to the side of base chassis 110a prior to exiting towards nose chassis 110b.

As described herein, device 100 can include one or more nuts configured to translate along various lead screws to control the articulation of device 100. For example, as shown, device 100 can include nuts 1461, 1672, 1612, and 1263. Nut 1461 can be configured to control the deployment of needle 142 (not shown but described herein). Nut 1672 can be configured control the pitch of nose chassis 110b relative to base chassis 110a. Nut 1612 can be configured to control the translation of nose chassis 110b relative to base chassis 110a. Nut 1263 can be configured to control the rotation of device 100 relative to base tissue attachment assembly 120a. In some embodiments, nut 1263 (e.g. and the associated lead screw) is configured to provide precise control of movement, such as when nut 1263 (e.g. and the associated lead screw) comprises a pitch of at least 10 threads per inch (TPI), such as at least 15, 25, 30, 40, and/or 50 TPI.

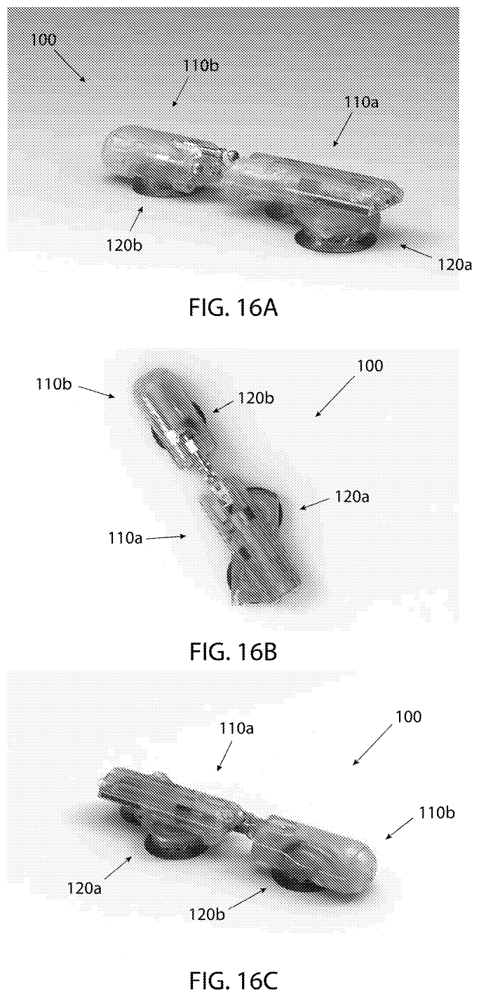

Referring now to FIGS. 16A-C, a back-perspective view, a top view, and a front-perspective view of an embodiment of a treatment device are illustrated, respectively, consistent with the present inventive concepts. Device 100 can be of similar construction and arrangement, and include similar components, as device 100 described in reference to FIG. 6 and otherwise herein. Device 100 can comprise base chassis 110a including base tissue attachment assembly 120a, and nose chassis 110b including nose tissue attachment assembly 120b, such as have been described herein. In the embodiments of FIGS. 16A-C, one or more control assemblies (e.g. rotation control assembly 163 and/or articulation control assembly 167 not shown but described herein) can comprise assemblies constructed and arranged to be manipulated by one or more steering cables, as described herein. Alternatively or additionally, one or more control assemblies of device 100 can be configured to be manipulated by a lead screw or other drive mechanism, also as described herein. In some embodiments, base tissue attachment assembly 120a is configured to rotate relative to base chassis 110a. Additionally or alternatively, nose chassis 110b can be configured to extend and/or retract relative to base chassis 110a.

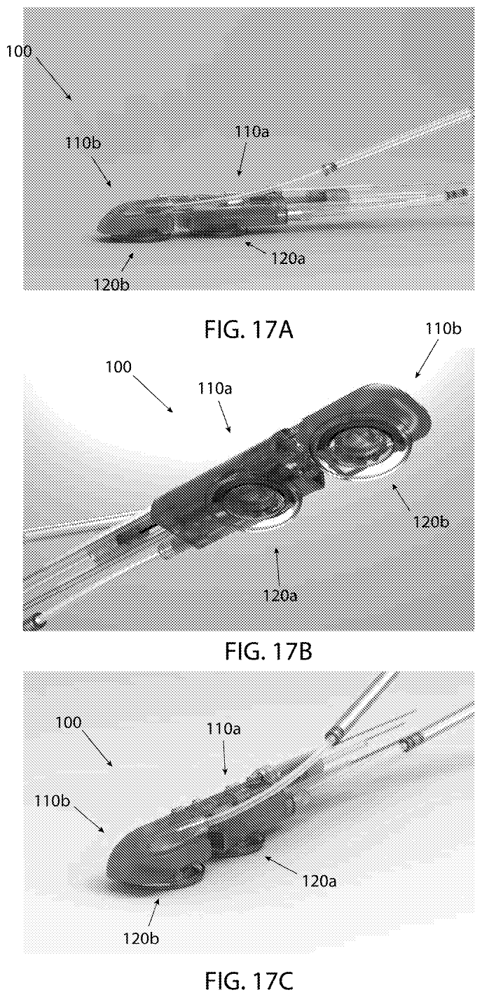

Referring now to FIGS. 17A-C, a side view, a bottom-perspective view, and a front-perspective view of another embodiment of a treatment device are illustrated, respectively, consistent with the present inventive concepts. Device 100 can be of similar construction and arrangement, and include similar components, as device 100 described in reference to FIG. 6 and otherwise herein. In the embodiments of FIGS. 17A-C, one or more control assemblies (e.g. rotation control assembly 163 and/or articulation control assembly 167 not shown but described herein) can comprise assemblies constructed and arranged to be manipulated by one or more steering cables, as described herein. Alternatively or additionally, one or more control assemblies of device 100 can be configured to be manipulated by a lead screw or other drive mechanism, also as described herein. In some embodiments, nose chassis 110b can be configured to articulate about a yaw axis relative to base chassis 110a. Additionally or alternatively, nose chassis 110b can be configured to extend from and/or pitch relative to base chassis 110a.

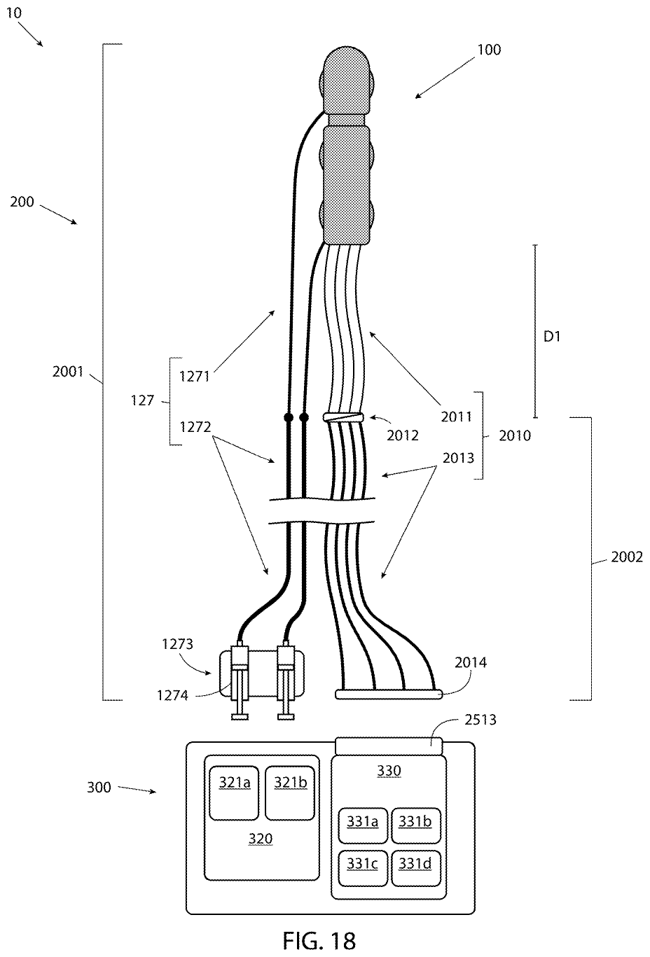

Referring now to FIG. 18, a schematic view of a treatment system illustrating portions of a treatment device, interface assembly, and console is illustrated, consistent with the present inventive concepts. System 10, device 100, interface assembly 200, and/or console 300 can be of similar construction and arrangement to similar components described in reference to FIG. 1, FIG. 6, and otherwise herein. In some embodiments, system 10 comprises a system with one or more disposable (“single use”) components, one or more components to be used multiple times (e.g. a “limited reuse component” for reuse no more than 10, 50, or 100 times), and one or more components constructed and arranged to be used repeatedly for an extended period (e.g. capital equipment). In some embodiments, device 100 is a disposable device, intended to be used in a single clinical procedure, and then disposed (e.g. discarded as medical waste or returned to the manufacture of system 10 for disposal, remanufacture, and/or analysis). In some embodiments, at least a portion of interface assembly 200 is a disposable component of system 10, also intended for a single clinical use. In some embodiments, at least a portion of interface assembly 200 is a limited reuse component, such as a component intended for use more than once (e.g. intended for use in more than one clinical procedure with more than one patient). In some embodiments, such limited reuse components can be constructed and arranged to be sterilizable between uses, such as via an autoclave sterilization process. In some embodiments, console 300 is intended for use over an extended period of time (e.g. the life of system 10 as it is used at a clinical site). In some embodiments, console 300 is configured to be upgraded and/or repaired if needed, such as by a technician enlisted by the manufacture of system 10. In some embodiments, console 300 is constructed and arranged to interface with various different models (e.g. different configurations) of device 100, such as when device 100 comprises a first device 100a with a first set of features or design functionalities, and a second device 100b with a second, different set of features or design functionalities (e.g. when a device 100a and a device 100b are configured to be used in different clinical procedures with different clinical goals).

In the embodiment illustrated in FIG. 18, device 100 is operably attached to multiple control conduits 2010. Control conduits 2010 can include control conduits 162, 164, 168, 141, and/or other control conduits as described herein. Control conduits 2010 can comprise conduits configured to translate rotational forces to device 100, such as when a conduit 2010 includes one or more torque cables. Conduits 2010 can operably attach one or more control assemblies of device 100 to a rotating control, such as a control provided by articulation control module 330 of console 300. Additionally, device 100 can be operably attached to multiple attachment conduits 127. Attachment conduits 127 can comprise conduits configured to fluidically attach one or more attachment assemblies of device 100 to a source of vacuum, such as a source of vacuum provided by attachment control module 320 of console 300. In some embodiments, control conduits 2010 comprise mechanical linkages (e.g. pull wires) configured to operably attach a portion of device 100 to a translating mechanism of console 300 (e.g. when articulation control module 330 is configured to push and/or pull the mechanical linkages).

In some embodiments, conduits 2010 and/or conduits 127 each comprise at least two portions. For example, each conduit 127 (two shown) can comprise a first portion 1271 extending proximally from device 100 as shown, and an associated second portion 1272 extending proximally from the proximal end of the first portion 1271, also as shown. In some embodiments, one or more first portions 1271 and their associated second portions 1272 are attached via a butt weld or other joint allowing a lumen passing therethrough to be maintained (e.g. without loss of integrity). Alternatively or additionally, one or more first portions 1271 and their associated second portions 1272 can be attached via mating connectors, such as mating luer connectors. As another example, each conduit 2010 (four shown) can comprise a first portion 2011 extending proximally from device 100 as shown, and an associated second portion 2013 extending proximally from the proximal end of the first portion 2011, also as shown. In some embodiments, one or more first portions 2011 and their associated second portions 2013 are operably attached via one or more mechanical couplings, connector 2012. In some embodiments, a single connector 2012 can be constructed and arranged to mate the first and second portions of two or more conduits 2010, such that rotational force is passed from the respective first portions to the respective second portions of each conduit 2010 being connected by connector 2012. In some embodiments, first portions 1271 and/or 2011 comprise a lower bending stiffness than the respective second portions 1272 and/or 2013 (e.g. a difference in bending stiffness of at least 5%, 10%, and/or 20%). In some embodiments, the first portions 1271 and/or 2011 extend proximally a distance D1 from device 100. Distance D1 can comprise a distance of at least 25 cm, and/or 50 cm. In some embodiments, first portions 1271 comprise vacuum tubing with an inner diameter of at least 0.25 mm and no more than 2 mm, such as approximately 0.5 mm. In some embodiments, first portions 2011 each comprise a torque coil with an outer diameter of no more than 1 mm. In some embodiments, portions 2013 and/or 1272 are constructed and arranged to extend into a sterile field, operably connecting console 300 (outside the sterile field) to device 100 (inside the sterile field).

In some embodiments, conduits 127 attach to syringe assembly 1273. Syringe assembly 1273 can comprise one, two, or more syringes, such as two syringes 1274 shown, where each syringe 1274 can be configured to attach to a conduit 127. In some embodiments, syringes 1274 of syringe assembly 1273 comprise multiple syringes of similar designs. Alternatively, each syringe 1274 of syringe assembly 1273 can comprise a syringe selected to provide a vacuum force specific to an associated attachment mechanism. For example, in some embodiments, base tissue attachment assembly 120a (illustrated in FIG. 9A) comprises a greater chamber volume than nose tissue attachment assembly 120b (illustrated in FIG. 14). As such, a syringe 1274 configured to attach to base tissue attachment assembly 120a can comprise a larger diameter and/or otherwise longer stroke volume than a syringe 1274 configured to attach to nose tissue attachment assembly 120b (e.g. a diameter of syringe 1274 that attaches to the assembly 120a that is at least 5%, 10%, and/or 20% larger that the diameter of a syringe 1274 that attaches to assembly 120b). In some embodiments, attachment control module 320 comprises one or more actuating mechanisms, syringe pumps 321a,b shown, for example one syringe pump 321 for each syringe 1274 of syringe assembly 1273. Syringe assembly 1273 can be constructed and arranged to removably attach to attachment control module 320.

In some embodiments, conduits 2010 terminate proximally at connector 2014. In some embodiments, connector 2014 comprises a single connector constructed and arranged to operably attach multiple conduits 2010 to articulation control module 330. Alternatively, connector 2014 can comprise multiple connectors, for example one connector for each conduit 2010. Articulation control module 330 can comprise connector 2513, configured to operably attach to connector 2014. In some embodiments, articulation control module 330 comprises one or more motion control mechanisms, motors 331a-d shown. In some embodiments, articulation control module 330 comprises one motor for each control assembly of device 100.

In some embodiments, interface assembly 200 comprises a first portion, portion 2001, that is pre-attached to device 100 (e.g. attached in a manufacturing process). Portion 2001 can include conduits 127 (e.g. including associated first portions 1271 and second portions 1272). Additionally, portion 2001 can include syringe assembly 1273. Portion 2001 can include first portions 2011 of conduits 2010, including a first portion of connector 2012 fixedly attached to first portions 2011. In some embodiments, portion 2001 is disposable, as described herein. In some embodiments, interface assembly 200 comprises a second portion, portion 2002, that is attachable to device 100 (e.g. during a clinical procedure). Portion 2002 can include second portions 2013 of conduits 2010, including a portion of connector 2012 and connector 2014. In some embodiments, portion 2002 is reusable, as described herein.

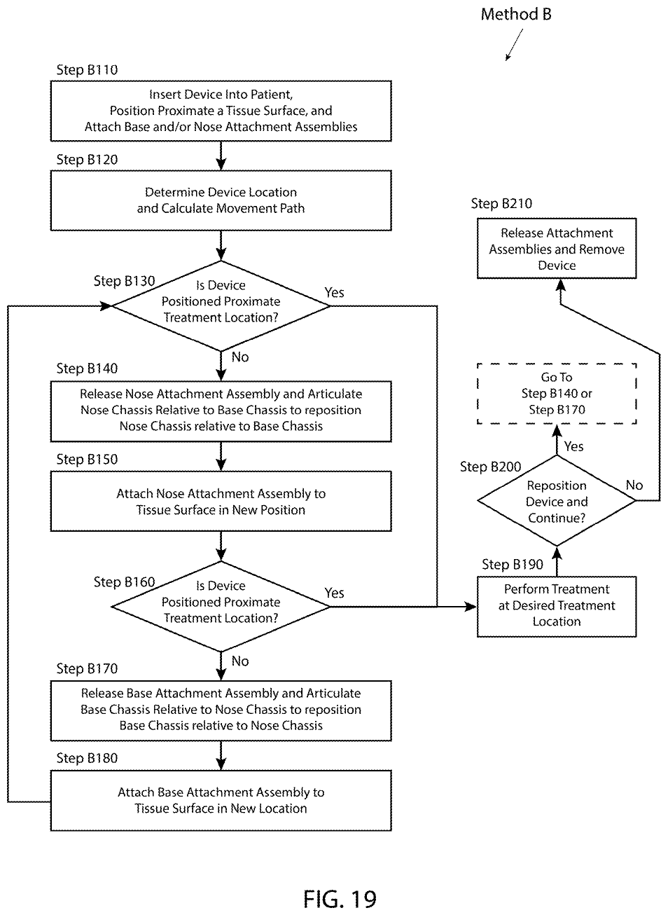

Referring now to FIG. 19, a flowchart of a method of maneuvering a device to a desired tissue location is illustrated, consistent with the present inventive concepts. Method B describes inserting a device, such as device 100 described herein, into a patient, maneuvering the device to a desired tissue location, and performing a procedure at the desired location. In Step B110, device 100 is inserted into the patient and positioned proximate a tissue surface. For example, when treating cardiac tissue, device 100 can be inserted into the chest of the patient, through the pericardium, and placed onto the epicardial surface, for example, near the apex of the heart. The epicardial surface can be accessed through a minimally invasive subxiphoid incision. Once placed proximate the tissue surface, either or both base tissue attachment assembly 120a and/or nose tissue attachment assembly 120b can be attached to the tissue surface to secure device 100 to the tissue surface. For example, and as described herein, suction can be applied to the tissue surface via grippers 125a and 125b to securely attach each of attachment assemblies 120a and 120b to the tissue surface.

In Step B120, system 10 (e.g. via algorithm 315) determines the location of device 100 relative to a desired treatment location and calculates a movement path along which to maneuver device 100 from the current location to the treatment location. Algorithm 315 can determine a set of movement operations (e.g. one or more translations and/or articulations between base chassis 110a and nose chassis 110b) to maneuver device 100 along the determined movement path. In some embodiments, system 10 is configured to automatically maneuver device 100 along at least a portion of the movement path, such as by robotically or otherwise automatically performing one or more of the movement operations as determined by algorithm 315. Alternatively or additionally, an operator of system 10 can manually (e.g. via one or more user controls of system 10) articulate the two chassis of device 100 relative to each other to reposition device 100. In some embodiments, device 100 can be manually or semi-automatically maneuvered while the position of device 100 relative to the patient is displayed to the operator via display 410 (e.g. via fluoroscopic and/or ultrasonic imaging).

In Step B130, if device 100 is located in a position such that a treatment assembly of device 100 (e.g. needle assembly 140) is sufficiently proximate (e.g. within 1 mm, 2 mm, 3 mm, and/or 5 mm) a desired tissue location to perform a treatment, method A continues to Step B190 and the treatment is performed. In some embodiments, immediately following insertion (e.g. in the inserted location), a first treatment step is performed, such as a cardiac mapping procedure step, before device 100 is repositioned to the next desired tissue location. In some embodiments, before, during, and/or after each maneuver of device 100, a treatment step can be performed. If device 100 is not in the desired treatment location, or a treatment step has been performed and an additional maneuver is desired, method B continues to Step B140.

In Step B140, nose attachment assembly 120b is released from the tissue (e.g. vacuum is released as described herein), and nose chassis 110b is articulated relative to base chassis 110a to reposition nose chassis 110b along the movement path. Subsequently, in Step B150, nose attachment assembly 120b is reattached to the tissue surface (e.g. vacuum is applied as described herein).

In Step B160, if device 100 is located in a position such that a treatment assembly of device 100 (e.g. needle assembly 140) is proximate a desired treatment location, method B continues to Step B190 and the desired treatment is performed. Otherwise, method B continues to Step B170.

In Step B170, base tissue attachment assembly 120a is released from the tissue, and base chassis 110a is articulated relative to nose chassis 110a to reposition base chassis 110b along the movement path. For example, base chassis 110a can be advanced towards nose chassis 110b (e.g. in an inch worm type manner), such that in a subsequent step, nose chassis 110b can be repositioned further along the movement path. Subsequently, in Step B180, base tissue attachment assembly 120a is reattached to the tissue surface. After base tissue attachment assembly 120a has been repositioned, method B returns to Step B130. Steps B130 through B180 are repeated until a treatment assembly of device 100 (e.g. needle assembly 140) is positioned proximate a desired treatment location.

Once device 100 has followed the determined movement path and is positioned proximate the desired treatment location, a treatment is performed in Step B190 at the desired treatment location. In Step B200, if subsequent treatment procedures are to be performed, method B returns to either Step B140 or B170, to reposition base chassis 110a or nose chassis 110b, respectively. Otherwise, if the procedure is complete, in Step B210 both attachment assemblies 120 can be released from the tissue and device 100 can be removed from the patient. In some embodiments, device 100 is maneuvered along a movement path back to the original insertion location before removal from the patient.

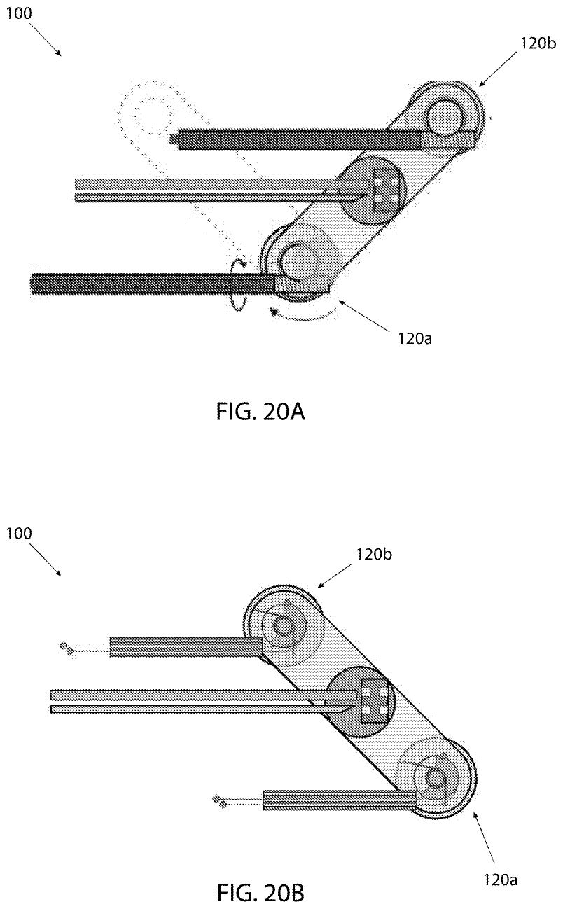

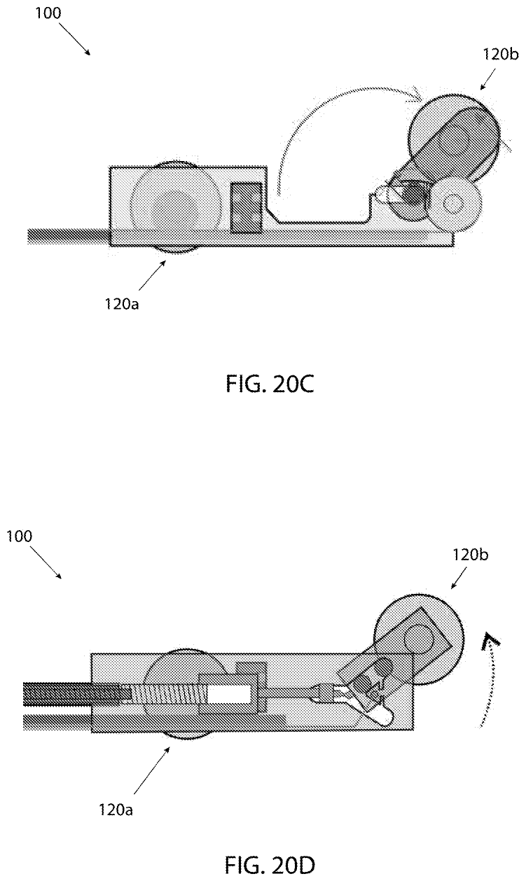

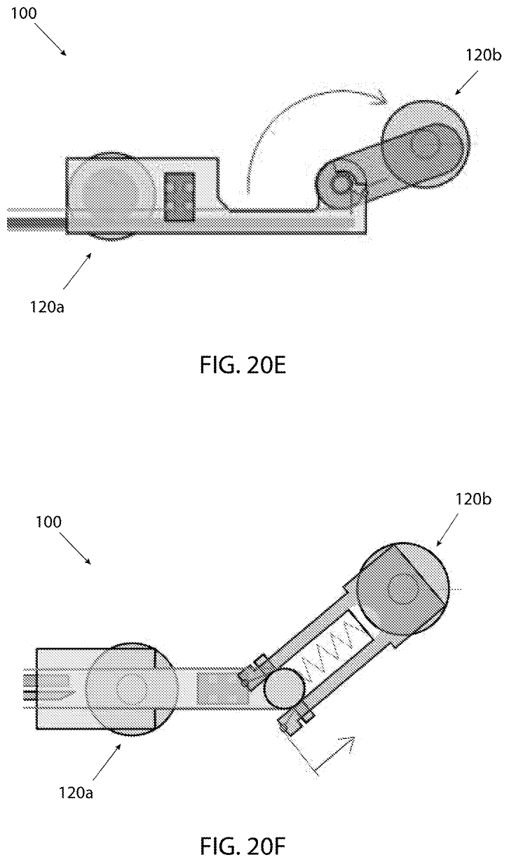

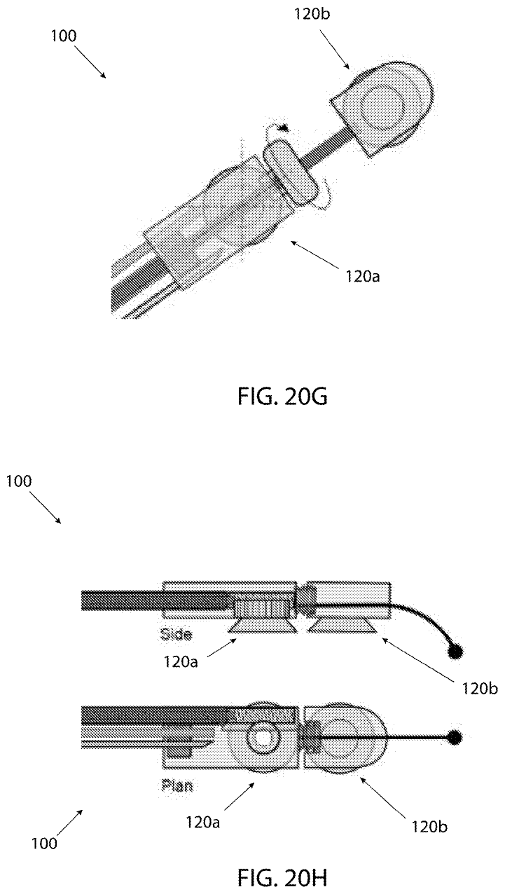

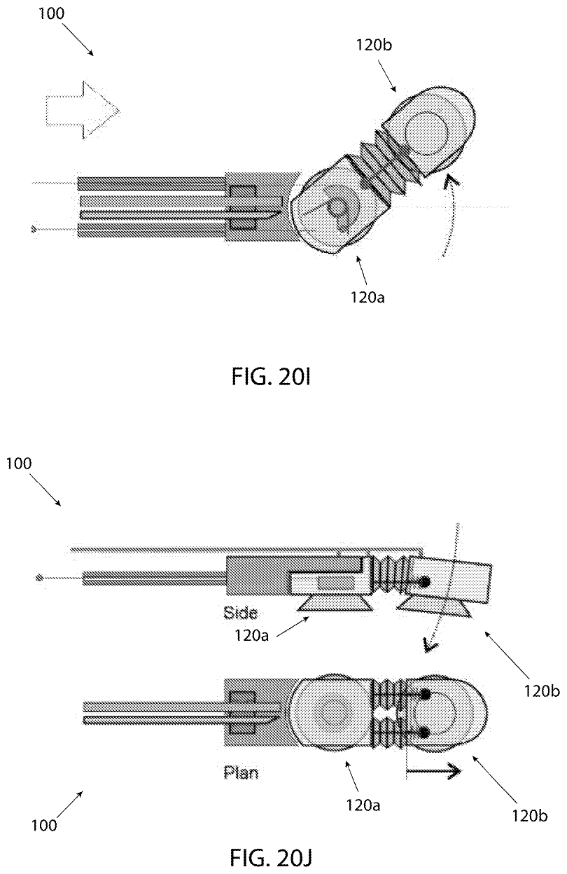

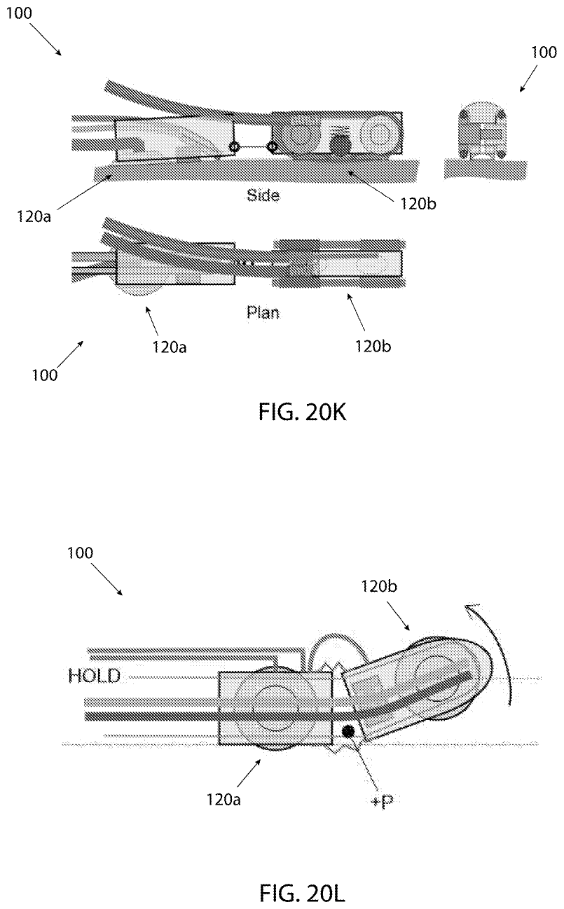

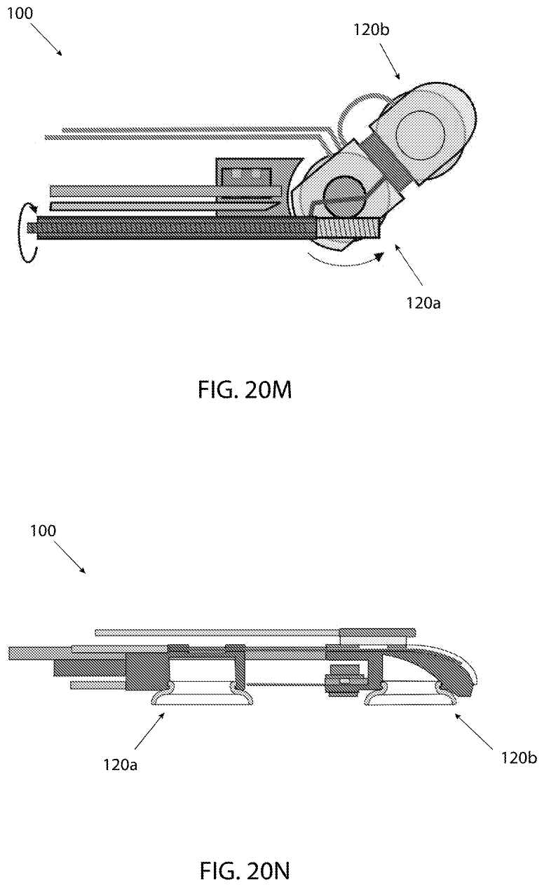

Referring now to FIGS. 20A-O, various views of various embodiments of a treatment device are illustrated, consistent with the present inventive concepts. The devices illustrated can include similar components to device 100 described in reference to FIG. 1, FIG. 6, and elsewhere herein. Devices 100 of FIGS. 20A-200 can each include at least two tissue attachment assemblies, attachment assemblies 120a and 120b shown. In some embodiments, at least a portion of device 100 is configured to rotate relative to attachment assembly 120a and/or attachment assembly 120b. In some embodiments, one or more control assemblies (e.g. rotation control assembly 163 and/or articulation control assembly 167 not shown but described herein) can comprise one or more assemblies constructed and arranged to be manipulated by one or more steering cables, as described herein. Alternatively or additionally, one or more control assemblies of device 100 can be configured to be manipulated by a lead screw or other drive mechanism, also as described herein. One or more portions of each device 100 of FIGS. 20A-200 can be configured to articulate, actuate, rotate, and/or otherwise move relative to another portion of the device, for example as indicated by the various arrows shown, and as described herein in reference to similar assemblies of the present inventive concepts.

Referring now to FIGS. 21A and 21B, perspective sectional views of various embodiments of a gripper for securing a treatment device to tissue are illustrated, consistent with the present inventive concepts. Grippers 125 can be of similar construction and arrangement to other grippers 125 described herein. Grippers 125 each comprise a flexible radial projection, skirt 1252, extending from walls 1251. Skirt 1252 can be configured to engage a tissue surface to create a seal between the tissue surface, and an associated chamber (e.g. a chamber of an attachment assembly to which gripper 125 is attached). In some embodiments, skirt 1252 extends radially and downward (e.g. away from the bottom of walls 1251) toward tissue. In some embodiments, skirt 1252 comprises a curled edge, such as an edge that curls upward away from tissue, as illustrated in FIG. 21B. In some embodiments, the curled edge of skirt 1252 can be configured to cause gripper 125 to tend to slide over a tissue surface, such as to avoid the edge of skirt 1252 from: folding under; frictionally engaging tissue (e.g. binding on the tissue surface); and/or otherwise undesirably interacting with the tissue surface.

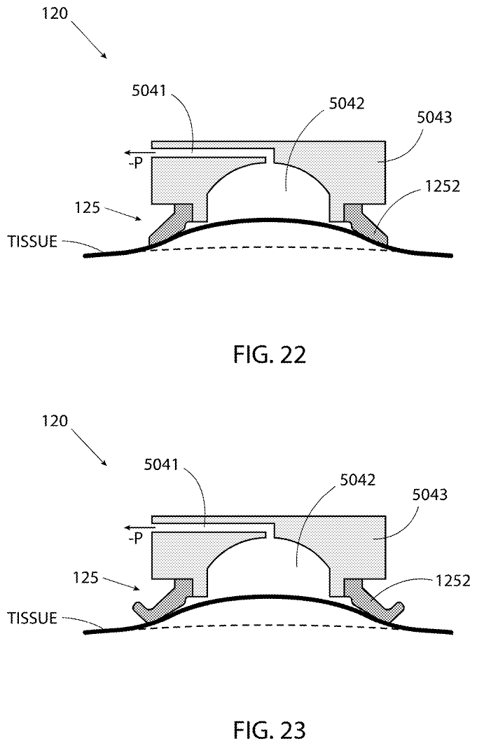

Referring now to FIG. 22, a sectional view of a tissue attachment assembly is illustrated, consistent with the present inventive concepts. Tissue attachment assembly 120 can be of similar construction and arrangement and comprise similar components to other tissue attachment assemblies 120 described herein. Assembly 120 comprises housing 5043 surrounding chamber 5042. Chamber 5042 can be configured to receive a vacuum (e.g. from console 300 of system 10 described herein) to cause assembly 120 to attach to tissue. Assembly 120 can include gripper 125 configured to operably engage the tissue. Gripper 125 includes a radial projection, skirt 1252, extending from housing 5043. Gripper 125 of FIG. 22 can be similar to gripper 125 illustrated in FIG. 21A. Assembly 120 can include lumen 5041, fluidly attached to a source of vacuum, for example provided by attachment control module 320. When vacuum pressure −p is applied to chamber 5042 via lumen 5041, gripper 125 can form a seal with the tissue, attaching assembly 120 to the tissue. In some embodiments, the tissue is partially drawn into chamber 5042 via the vacuum as shown.

Referring additionally to FIG. 23, a sectional view of a tissue attachment assembly is illustrated, consistent with the present inventive concepts. Tissue attachment assembly 120 can be of similar construction and arrangement and comprise similar components to other tissue attachment assemblies 120 described herein. Gripper 125 of FIG. 23 can be similar to gripper 125 of FIG. 21B.

Referring now to FIGS. 24A and 24B, sectional views of a tissue attachment assembly are illustrated, consistent with the present inventive concepts. Tissue attachment assembly 120 can be of similar construction and arrangement and comprise similar components to other tissue attachment assemblies 120 described herein. Assembly 120 comprises housing 5043 surrounding chamber 5042. Chamber 5042 can be configured to receive a vacuum (e.g. from console 300 of system 10 described herein) to cause assembly 120 to attach to tissue. Assembly 120 can include lumen 5041, fluidly attached to a source of vacuum, for example vacuum provided by attachment control module 320. Assembly 120 can include gripper 125 which can be configured to operably engage with tissue. Gripper 125 includes a radial projection, skirt 1252, extending from housing 5043. In some embodiments, gripper 125 includes a flexible substrate, membrane 5055, configured to seal chamber 5042. In some embodiments, when positive pressure+P shown is applied to chamber 5042 (e.g. via lumen 5041), membrane 5055 is configured to deform away from chamber 5042 and towards tissue, as illustrated by the dashed lines in FIG. 24A. When negative pressure −P shown is applied to chamber 5042 (e.g. via lumen 5041), membrane 5055 is pulled into chamber 5042, creating an area of negative pressure between membrane 5055 and the tissue, attaching assembly 120 to the tissue. In some embodiments, the tissue is partially drawn into chamber 5042 as shown in FIG. 24B. In some embodiments, lumen 5041 enters chamber 5042 proximate the outer edge of the chamber, such as to reduce the likelihood of membrane 5055 occluding lumen 5041 when vacuum is applied. Additionally or alternatively, chamber 5042 can comprise one or more recesses (e.g. one or more channels) along its inner surface extending from lumen 5041, the recesses configured to reduce the likelihood of membrane 5055 occluding lumen 5041 when vacuum is applied.

In some embodiments, console 300 is configured to manipulate a liquid (e.g. saline) within chamber 5042, for example using a closed fluid pathway between one or more syringes and chamber 5042, to control the position of membrane 5055. For example, console 300 can draw liquid from chamber 5042 to pull membrane 5055 into the chamber.

Referring now to FIGS. 25A and 25B, sectional views of a tissue attachment assembly are illustrated, consistent with the present inventive concepts. Tissue attachment assembly 120 can be of similar construction and arrangement and comprise similar components to tissue attachment assemblies 120 described in reference to FIGS. 24A and 24B and otherwise herein. In some embodiments, assembly 120 comprises a linkage, control cable 5044, extending through lumen 5041 and operably attaching (e.g. mechanically attaching) to membrane 5055. In some embodiments, when control cable 5044 is advanced, membrane 5055 is configured to deform in a direction away from chamber 5042 and toward tissue, as illustrated by the dashed lines in FIG. 25A. When control cable 5044 is retracted, membrane 5055 is pulled into chamber 5042, creating an area of negative pressure between membrane 5055 and the tissue, attaching assembly 120 to the tissue. In some embodiments, the tissue is partially drawn into chamber 5042 as shown in FIG. 25B.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for treating and/or diagnosing tissue from a tissue surface, the system comprising:
a console;
an interface assembly; and
a mobile device configured to navigate the tissue surface, the mobile device comprising:
a first tissue attachment assembly;
a second tissue attachment assembly; and
at least one articulation assembly;
wherein the interface assembly operably connects the console to the mobile device;
wherein the console is configured to manipulate the at least one articulation assembly via the interface assembly;
wherein the at least one articulation assembly is configured to navigate from a first tissue location on the tissue surface to a second tissue location on the tissue surface by:
at least one time performing a first movement by manipulating the position of the first tissue attachment assembly relative to the tissue surface while the second tissue attachment assembly is attached to the tissue; and
at least one time performing a second movement by manipulating the position of the second tissue attachment assembly relative to the tissue surface while the first tissue attachment assembly is attached to tissue;
and
wherein the mobile device is configured to diagnose and/or treat tissue proximate the second tissue location.

2.-22. (canceled)
